(19) United States
(12) Patent Application Publication
(10) Pub. No.: US 2009/0319011 A1
(43) Pub. Date: Dec. 24, 2009

(54) METHOD AND SYSTEM FOR WARMING OR COOLING A FLUID

(76) Inventor: Keith Rosiello, Shrewsbury, MA (US)

Correspondence Address: FOLEY & LARDNER LLP, 111 HUNTINGTON AVENUE, 26TH FLOOR, BOSTON, MA, 02199-7610, US

(21) Appl. No.: 12/161,490

(22) PCT Filed: Jan. 19, 2007
(86) PCT No.: PCT/US07/01510

§ 371 Date: May 1, 2009

Related U.S. Application Data
(60) Provisional application No. 60/760,771, filed on Jan. 19, 2006.

(51) Int. Cl. A61F 7/00; F28D 7/10; H05B 1/02; F16K 31/00; F24H 1/10

(52) U.S. Cl. 607/105; 392/496; 219/494; 137/468; 392/466

(57) ABSTRACT

The invention is directed generally to a method and system for controlling the temperature of a fluid, i.e., warming or cooling a fluid, and more particularly, to a method and system for warming a fluid to be delivered to the body of a patient. In a preferred embodiment, a method and system for warming a fluid to be delivered into the body of a patient is provided and may include a controller and a fluid delivery-line.

### FIELD OF THE INVENTION

The invention is directed generally to a method and system for controlling the temperature of a fluid, e.g., warming or cooling a fluid, and more particularly, to a method and system for warming a fluid to be delivered to the body of a patient.

### BACKGROUND OF THE INVENTION

Thermoregulatory mechanisms exist in the healthy mammalian body to maintain the body temperature within a narrow range. For example, the human body is maintained at a constant temperature of about 98.6° F. (37° C.). The normal temperature “set-point” of the mammalian body, however, may vary between different mammals. The maintenance of the body at a normal “set-point” is generally a desirable condition and is called normothermia.

For various reasons, e.g., environmental exposure or blood loss, an individual may develop a body temperature that is below the normal temperature “set-point,” a condition known as hypothermia. In contrast, in a condition known as hyperthermia, an individual develops a body temperature that is above the normal temperature “set-point.” For example, hyperthermia may be caused by environmental exposure or infection. In the human, these conditions are generally harmful to an individual and are usually treated to reverse the condition and return them to normothermic status. In certain other situations, however, these conditions may be desirable and may even be intentionally induced. Indeed, in some clinical circumstances, it is desirable to alter the overall temperature of the body, while under other circumstances it is desirable to alter the temperature of a specific body region or tissue. See generally, US Patent application US 2003/0195597, published Oct. 16, 2003 and incorporated by reference herein in its entirety.

Various medical items (e.g., surgical tools, bottles, bags) and solutions (e.g., whole blood, blood serum, saline, antibiotics or other drugs, intravenous solutions) require heating to a selected temperature prior to use in a medical procedure. Most parenteral fluids, such as saline, are commonly stored at “normal room temperature” generally considered 65-75° F. (18.3-23.9° C.). Other parenteral fluids, such as whole blood, are stored refrigerated at a temperature of 39.2° F. (4° C.). Yet other parenteral fluids are cryopreserved and, due to time constraints, often only uniformly thawed just enough to allow fluid flow. It is advantageous for intravenously administered parenteral fluids to be warmed to near normal body temperature to prevent insult to the patient and, in hypothermia-related cases, reduce the level of trauma.

A number of systems and methods have been designed to address the need to alter the temperature of parenteral fluids, e.g., warm parenteral fluids, for use in transfusion medicine. Most common are bulk fluid warmers. These devices warm a bulk volume of fluid such as a bag of whole blood using a reservoir of heated fluid, the fluid usually being water. The bag of fluid to be warmed is doubled bagged for safety and immersed in the heated bath while being constantly mixed to insure uniform heating. After some time, usually 10-40 minutes depending on the starting and desired fluid temperatures, the fluid is ready to be transfused.

Other prior art devices include in-line warmers, which are used to warm fluids for use in transfusion medicine. These devices use various heating techniques to warm fluids as they flow from the supply bag to the patient. The heating techniques vary greatly, e.g., U.S. Pat. No. 5,690,614 uses microwave energy, U.S. Pat. No. 5,807,332 uses a heated stream of air, and U.S. Pat. No. 5,101,804 uses a chemical reaction. Other prior art references use electrically heated plates in either direct or indirect contact with the fluid to be warmed.

The methods and systems available to suitably warm fluids have several limitations in common. One of the common problems associated with current fluid warmers (a.k.a., “blood warmers”) is the lack of portability, in particular the need for an AC power source, or a large, cumbersome battery. Another common problem with current fluid warmers is the lack of flexibility to specific environments such as ambulances, emergency rooms and field use. Yet another common problem of current fluid warmers relates to fluid flow-rate limitations and associated localized overheating of fluid due to serpentine fluid pathways, or the inefficient application of heat to the fluid.

Finally, much of the prior art is designed to be a modular component within the total intravenous administration set (hereinafter, “I.V. set”). This often requires the use of a pre-warmer I.V. set as well as a post-warmer I.V. set to warm a fluid. These I.V. sets may need to be several feet long to accommodate the spatial logistics of a surgical procedure, or the high level of activity in an emergency room. The post-warmer I.V. set is a source of significant heat loss, creating a varying temperature differential between the fluid warmer and the patient. Furthermore, the need for I.V. sets is not preferred for portability and field use.

There is a need for a method and portable system for warming a fluid, in particular, a fluid to be delivered into the body of a patient, that is both adaptable to field use (e.g., healthcare settings), and minimizes the temperature differential between the fluid warmer and the patient.

### SUMMARY OF THE INVENTION

The system and method of the present invention overcome the above-noted problems and concerns, and some embodiments of the present invention provide a novel fluid warmer for delivering a fluid, medicinal or otherwise, to the body of a patient. In other embodiments, the present invention provides a novel fluid cooler for delivering a fluid to the body of a patient. A fluid(s) delivered by the method and system of the invention can be blood-based fluids or non-blood-based fluids, that include but are not limited to, e.g., whole blood, blood serum, saline, cryopreservant, antibiotics or other drugs. A patient may include any living organism, especially mammal, and in particular, humans. The method and system of the invention is useful to deliver fluid into the body of a patient, e.g., but not limited to, intravenous or intraperitoneal routes. The method and system of the invention can also be used in combination with other heat exchange devices, e.g., heat exchange catheters. The method and system of the present invention is useful to provide warming of a specific region or tissue of a patient.

The invention described herein overcomes the aforementioned limitations by integrating an I.V. set with a novel warming method and system, for example. In one embodiment of the present invention, the novel method and system may use a variety of power sources from AC to a small battery of both rechargeable and disposable types. The method and system may also include a delivery-line component between the fluid supply bag and the patient connection. The total length of the delivery-line component may be comprised of a uniform tube construction to warm the fluid along its entire length. In another embodiment of the present invention, the delivery-line component is a multiple tube construction joined by mechanical union fittings. In yet another embodiment of the present invention the delivery-line component is a multiple tube construction joined by a direct material bonding.

Accordingly, this novel design according to some embodiments of the present invention may allow the fluid delivery pathway to be flexible, non-kinking, in lengths of one foot and greater. The choice of power sources and the ability of the fluid warmer to act as an I.V. set enable some of the embodiments of the present invention well suited to portability and use in a variety of environments. Gradual and efficient warming over the entire non-serpentine fluid delivery length, for example, may support low and high (1 mL/min to 600 mL/min) flow rates for a variety of parenteral fluids, including whole blood, substantially eliminating or limiting damage to the fluid and/or patient, or overheating.

Thus, the new design according to some embodiments of the present invention may provide a fluid warmer that is portable, adaptable to different environments and easy to use.

Accordingly, in one embodiment, the present invention provides a system for heating a fluid for delivery into a body of a patient which includes a flexible fluid supply reservoir, and a controller and a fluid delivery-line wherein the delivery-line includes a fluid delivery tube for communicating a fluid; a heating element positioned proximate a surface of the fluid delivery-line to heat fluid within the tube; a first thermal sensor positioned on the exterior of the fluid delivery tube and adjacent to the fluid supply reservoir; and a second thermal sensor positioned at a heater assemble outlet and in communication with the lumen of the fluid delivery tube. The fluid delivery-line of the system may also include a third thermal sensor positioned at a heater assembly inlet and in communication with a lumen of the fluid delivery tube. The heating element can be spaced apart from an outer surface of the second tube. The wall of the tube can include a thermal medium for distributing heat received by the outer surface of the tube from the heating element. The system can include a heating element that surrounds the tube. The heating element can spirally surround the tube. The heating element can include a plurality of heating elements surrounding the tube and having a length positioned substantially parallel to a length of the tube. The heating element can include a plurality of heating elements, each circumferentially surrounding the tube and spaced apart from one another along a length of the tube. The heating element can be surrounded by a thermal medium. The thermal medium can include a fluid. The fluid delivery tube can include a bag spike positioned at one end. The fluid delivery tube can include a transfusion needle and/or a leur-lock at one end. The heating element and/or thermal sensor can be in electrical contact with the controller. The controller can be connected to a power source. The power source can be a one-time use battery pack, a rechargeable battery pack, AC power, and DC power. The tube can be sterile prior to use. The controller can provide an electrical current to the heating element. The controller can control the temperature of the fluid delivery-line tube by sensing a temperature corresponding to a temperature of fluid within the fluid delivery-line and adjusting the amount of current supplied to the heating element to maintain the temperature of the fluid independent of the flow rate of the fluid. The system of the invention can include a heat element connector and/or a thermal sensor connector for connecting the heat element and thermal sensor, respectively, to corresponding connectors on the controller. The system can include a valve. The valve can be/include a temperature actuated valve that opens upon the temperature of the fluid within the second tube reaching a predetermined value. The system can include a metering means for determining a flow rate of fluid traversing through the fluid delivery tube. The system can include a heat-conductive member having a first portion placed adjacent an interior portion of the fluid delivery tube and a second portion placed proximate the heating element, wherein the heat-conductive material transfers heat from the heating element to the interior portion of the fluid delivery tube. The system can include an insulative tube, wherein the fluid delivery tube is positioned within the insulative tube. The system can include a thermal medium positioned between the fluid delivery tube and the insulative tube. The thermal medium can envelop the heating element.

In another aspect, the present invention provides a method of heating a fluid for delivery into the body of a patient which includes providing a fluid delivery tube having a first end for connection to a flexible fluid supply reservoir; a controller; and a fluid delivery-line where the fluid delivery-line includes a fluid source and a second end for delivering the fluid from the fluid source to a destination; where an electrical current is applied to a heating element proximate to and/or within the fluid delivery tube to heat fluid therein to a predetermined temperature; sensing, via a first thermal sensor positioned on the exterior of the fluid delivery tube and adjacent to a flexible fluid supply reservoir and a second thermal sensor positioned at a heater assemble outlet and in communication with the lumen of the fluid delivery tube, a temperature corresponding to the temperature of the fluid within the tube; and adjusting the current applied to heating element via the controller based upon the sensed temperature. The fluid delivery-line can further include a third thermal sensor positioned at a heater assembly inlet and in communication with a lumen of the fluid delivery tube. The current can be decreased or stopped upon the temperature of the fluid delivery tube reaching the predetermined temperature. The method can also include opening a valve which controls the movement of fluid from the fluid delivery-line to the patient upon the temperature of the fluid for delivery reaching the predetermined temperature. The method can also include sensing a flow-rate of the fluid being delivered to the patient.

In another embodiment, the invention provides a system for heating a fluid for delivery into the body of a patient including a flexible fluid supply reservoir; a controller; and a fluid delivery-line where the delivery-line includes an insulative tube; a fluid delivery tube positioned within the first tube, the fluid delivery tube for communicating a fluid; a fluid delivery-line having a first end for receiving fluid from a fluid source and delivering the fluid to a destination, a heating element positioned proximate a surface of the fluid delivery-line to heat fluid within the tube; a first thermal sensor positioned on the exterior of the fluid delivery tube and adjacent to the fluid supply reservoir; and a second thermal sensor positioned at a heater assemble outlet and in communication with the lumen of the fluid delivery tube; and a thermal medium positioned between the first tube and the second tube. The system can include a third thermal sensor positioned at a heater assembly inlet and in communication with a lumen of the fluid delivery tube.

In another aspect, the system of the present invention can be used for cooling a fluid. The heat element is replaced with a hollow tube for circulating a coolant or a solid metallic chilling element that serves to lower the temperature of the fluid in the delivery-line. This configuration may be used in the delivery of cooled fluid to a patient, for I.V. use and/or other fluid administration techniques. The configuration may also be used for controlling the temperature of a target tissue or the temperature of a patient.

In yet another aspect of the present invention, the system has both heating and cooling elements and can be used for warming and cooling, thereby controlling the temperature of a fluid, the temperature of a target tissue, or the temperature of a patient.

Details of the above-described embodiments of the present invention are expanded and discussed below with reference to figures for the present invention.

### BRIEF DESCRIPTION OF THE DRAWINGS

FIG. 1 is a schematic diagram of an overall fluid warming system according to some embodiments of the present invention.

FIG. 2 is a cross-section diagram illustrating a cross-section of a fluid delivery-line for use in a fluid warming system according to some embodiments of the present invention.

FIG. 3 is a perspective view of a schematic of a heating element according to some of the embodiments of the present invention.

FIGS. 4A-4D are each a perspective schematic diagram of a fluid delivery-line for use in a fluid warming system according to some of the embodiments of the invention.

FIG. 5A is a schematic perspective view of connectors for connecting elements of a fluid delivery-line to a controller in some embodiments of the present invention.

FIG. 5B is a schematic perspective view of a heat-conductive element of a fluid delivery-line as disclosed in some embodiments of the present invention.

FIG. 6 is a schematic diagram of a controller for use in a fluid warming system according to some embodiments of the present invention.

FIGS. 7A and 7B are block diagrams of systems according to some of the embodiments of the present invention.

FIG. 8 is a schematic diagram illustrating the flow of data and controls for the algorithm development process described in Example 1.

FIGS. 9A-9C are each a perspective schematic diagram of a fluid delivery-line for use in a fluid warming system according to some of the embodiments of the present invention.

FIG. 10 is a perspective schematic diagram of a fluid delivery-line for use in a fluid warming system according to some of the embodiments of the present invention.

FIGS. 11A-11C are each a perspective schematic diagram of a fluid delivery-line for use in a fluid warming system according to some of the embodiments of the present invention.

FIGS. 12A-12B are each a perspective schematic diagram of a mid-line fluid delivery-line assembly for use in a fluid warming system according to some of the embodiments of the present invention.

FIGS. 13A-13C are each a perspective schematic diagram of a mid-line fluid delivery-line assembly for use in a fluid warming system according to some of the embodiments of the present invention.

FIGS. 14A and 14B are each a perspective schematic diagram of an end-fitment for use in a fluid warming system according to some of the embodiments of the present invention.

FIGS. 15A-15C are each a perspective schematic diagram of an end-fitment for use in a fluid warming system according to some of the embodiments of the present irivention.

FIG. 16A is a perspective schematic diagram illustrating the positioning of a temperature sensor in a delivery-line component with outer lumens according to some embodiments of the present invention.

FIG. 16B is a perspective schematic diagram of an end-fitment assembly for use in a fluid warming system according to some of the embodiments of the present invention.

FIG. 17 is a perspective schematic diagram of an outer collar with mating-lock feature for use in a fluid warming system according to some of the embodiments of the present invention.

FIGS. 18A-18C are each a perspective schematic diagram of an end-fitment assembly for use in a fluid warming system according to some of the embodiments of the present invention.

FIG. 19 is a perspective schematic diagram of an end-fitment assembly for use in a fluid warming system according to some of the embodiments of the present invention.

FIG. 20A is a perspective schematic diagram of an in-stream temperature sensor gasket for use in a fluid warming system according to some of the embodiments of the present invention.

FIG. 20B is a perspective schematic diagram of a mid-stream temperature sensor gasket for use in a fluid warming system according to some of the embodiments of the present invention.

FIG. 20C is a perspective schematic diagram of an insulated temperature sensor gasket for use in a fluid warming system according to some of the embodiments of the present invention.

FIG. 21A is a perspective schematic diagram of a heater element wire connector with a spade-type terminal for use in a fluid warming system according to some of the embodiments of the present invention.

FIG. 21B is a perspective schematic diagram of a heater element wire connector with a connection terminal bent at a ninety-degree angle for use in a fluid warming system according to some of the embodiments of the present invention.

FIG. 21C is a perspective schematic diagram of a heater element wire connector with a crimp-type terminal for use in a fluid warming system according to some of the embodiments of the present invention.

FIG. 22A is a perspective schematic diagram of a heater element wire connector with crimp-style terminals for use in a fluid warming system according to some of the embodiments of the present invention.

FIG. 22B is a perspective schematic diagram of a heater element wire connector with push-lock-style terminals for use in a fluid warming system according to some of the embodiments of the present invention.

FIG. 23A is a perspective schematic diagram showing the placement of a heater element wire connector with a crimp-style terminal on a delivery-line component with exposed wires for use in a fluid warming system according to some of the embodiments of the present invention.

FIGS. 23B and 23C are each a perspective schematic diagram showing the placement of a heater element wire connector with push-lock-style terminals on a delivery-line component with exposed wires for use in a fluid warming system according to some of the embodiments of the present invention.

FIGS. 24A-24C are each a perspective schematic diagram showing the placement of a heater element wire connector with push-lock-style terminals on a delivery-line component with embedded wires for use in a fluid warming system according to some of the embodiments of the present invention.

FIG. 25A is a perspective schematic diagram of a center temperature sensor for use in a fluid warming system according to some of the embodiments of the present invention.

FIG. 25B is a perspective schematic diagram of a silicone-plug-embedded-temperature sensor for use in a fluid warming system according to some of the embodiments of the present invention.

FIG. 25C is a perspective schematic diagram of a push-pin-style temperature sensor for use in a fluid warming system according to some of the embodiments of the present invention.

FIG. 26A is a block diagram of a system according to some of the embodiments of the present invention.

FIG. 26B is a block diagram of a system according to some of the embodiments of the present invention.

FIG. 27 is a block diagram of a system according to some of the embodiments of the embodiments of the present invention.

FIG. 28 is a diagram of a controller panel of a system according to some of the embodiments of the present invention.

### DETAILED DESCRIPTION OF THE INVENTION

It will be understood that there are several advantages to using the method and system of the present invention to warm a fluid. For example, the method and system of the invention can improve patient comfort during infusion therapies by providing external warming of fluids prior to their administration to a patient. The method and system can protect against hypothermia in patients receiving a large volume of intravenous fluid, e.g., patients undergoing hemodiafiltration, hemodialysis, hemofiltration, or ultrafiltration. The method and system of the invention minimize the variation in temperature of fluid between the warming system and the patient and provides a portable system useful in a variety of environments. The method and system of the invention can protect against current leakage and subsequent electrocution of an individual, e.g., a patient, in contact with the system.

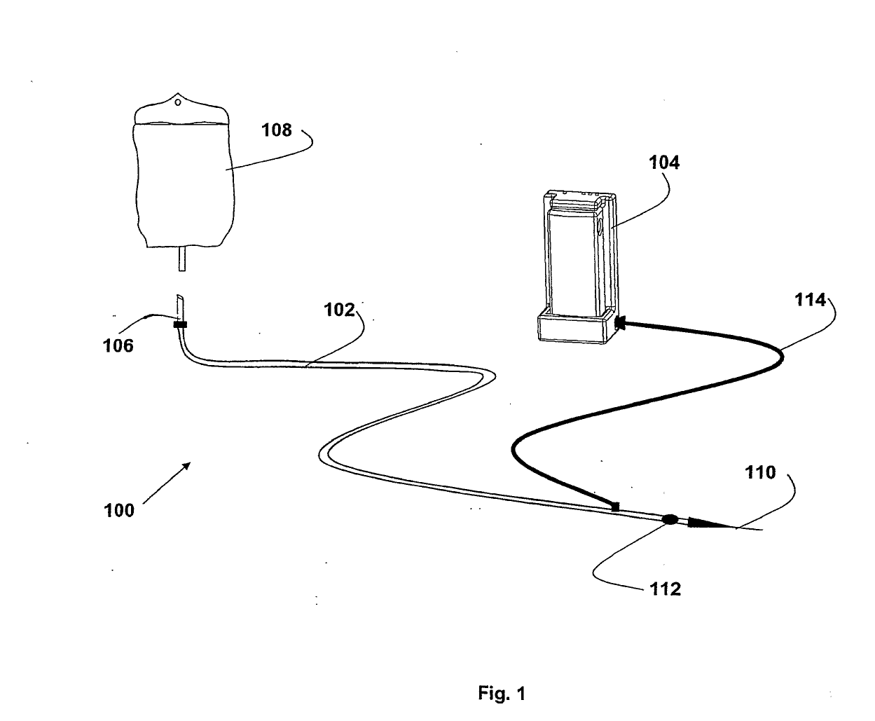

As shown in FIG. 1, some of the embodiments of the present invention include the following features. A fluid warming system 100 may include a fluid delivery-line 102 (a.k.a., fluid delivery tube) and a controller 104. The system may further include a bag spike 106 connected to one end of the fluid delivery-line which may be used to fluidly connect the fluid delivery-line to a container 108 (e.g., bag) of fluid for delivery to the body of a patient. Such bag spikes may include those disclosed in U.S. Pat. Nos. 5,445,630, 4,432,765 and 5,232,109, each of which is herein incorporated by reference in their entireties.

The controller 104 is connected to the fluid delivery-line via one or more wire based (or other communication devices/means) connection lines 114. The connection may be for supplying electrical current to a heating element and for getting a signal relating to a temperature indicative of the temperature of the fluid within the fluid delivery-line.

A transfusion needle 110 at the other end of the fluid delivery-line a transfusion needle may be connected thereto. The transfusion needle is inserted into, for example, a blood vessel of the patient, so that the fluid traversing through the fluid delivery-line may enter the body of the patient. In addition to the transfusion needles, luer-locks may be incorporated at an end of the fluid delivery-line. Such luer-locks may include, for example, U.S. Pat. Nos. 5,620,427, 5,738,144 and 6,083,194, herein incorporated by reference in their entireties.

In addition, customized end or union fitments may be incorporated at either end or one or more mid-line locations of the fluid delivery system.

Each of the bag spike, the transfusion needle, luer-lock, or custom fitment is preferably attached to the fluid delivery-line and form a sterile and/or airtight seal thereto. Moreover, in some embodiments, it is preferable that the fluid delivery-line be sterile or sterilized prior to use. In one embodiment of the present invention, the fluid delivery-line, bag spike and/or transfusion needle (preferably all together; the “fluid delivery-line system”), is a single use system that is sterilized upon manufacture and sealed in an airtight package. When used, the package is opened and the system (or individual components) connected to the fluid container and controller and used for delivering the fluid contained in the container to the body of a patient. After this single use, the fluid delivery-line system is disposed, preferably as medical waste.

A valve 112 may be positioned along the fluid delivery-line at any position, for controlling the flow of the fluid within the inner fluid delivery tube. In one embodiment, the valve is positioned adjacent the transfusion needle. The valve may be a mechanically and/or electrically actuated valve controlled by the controller, and may also may be a passively operated valve which may be actuated by a change in temperature of the fluid within the fluid delivery tube. In that regard, the valve may be made of a bi-metal material, that opens upon the temperature of the fluid reaching, predetermined temperature. In such an embodiment, the valve may be located at the end of the fluid delivery-line adjacent the transfusion needle. The valve may also be of the type that may be manually activated (either electronically or mechanically) by an individual (e.g., medical personnel).

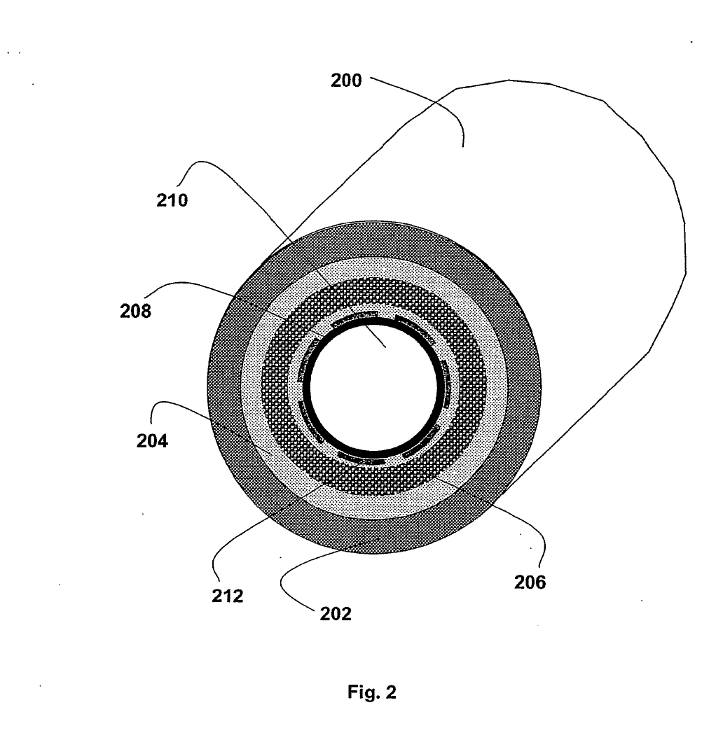

As shown in FIG. 2A, which illustrates a cross section of a fluid delivery-line 200 according to one embodiment of the invention, the fluid delivery-line may include the following components. An outer sleeve or tube of an insulation material (for example) 202 surrounds a thermal medium 204. Within the thermal medium a heating element 206 is provided which may surround a fluid delivery tube 208. The fluid delivery tube includes a sterile fluid pathway 210 for fluids which are warmed therein.

Positioned adjacent the wall of the fluid delivery tube is one or more thermal sensors 212. In this embodiment, the one or more thermal sensors sense a temperature of the fluid delivery tube. This temperature may be directly related to the temperature of the fluid within the fluid delivery tube. One or more thermal sensors, e.g., wire or probe-type sensors, may also be used to directly sense the fluid within the delivery tube via direct contact.

The outer sleeve may be constructed from any tubular form of application appropriate insulation material. Such material may include plastic and foam based materials made from, for example, polyethylene. The outer sleeve may also contain or be constructed from additional materials, such as silicon rubber or urethane formulations or custom blended thermoplastics, e.g., tygone. In another embodiment of the present invention, the outer sleeve component is constructed from a material that does not have properties of insulation. Where the outer sleeve is not constructed from material that has properties of insulation, the insulative function may be served by another components within the assembly.

The thermal medium may include a gas, liquid or solid, or a combination thereof, which allows heat produced by the heating element to be distributed more evenly. This is preferred since a direct application of the heat generated by the heating element to the wall of the inner tube, if the heating element is placed close to the wall of the inner fluid delivery tube, can damage or destroy the fluid being delivered by the system to the body of a patient (e.g., blood cells) since the amount of heat at the heating element may generally be higher.

Examples of the thermal medium may include air, water, saline and/or alcohol based solutions. Preferably, the thermal medium may also include ceramics, metals, plastics, natural fibers or some combination thereof. In some embodiments of the present invention, the thermal medium may be incorporated into the wall of the inner fluid delivery tube. In such an embodiment, the heating element may be positioned on the outer surface of the inner tube. The thermal medium wall thus evenly distributes the heat from the heating element to the non-heated portions of the inner fluid delivery tube and subsequently the fluid within the tube.

As shown in FIG. 2B, which illustrates a cross-section of a fluid delivery-line according to some embodiments of the invention, the fluid delivery-line may include the following components. A multi-lumen outer sleeve 231, in which each lumen 233 serves to contain a material, air for example, whose physical properties features both electric and thermal insulation is a component thereof. The lumen may also contain materials to assist with fluid heating or cooling functions. In some embodiments of the invention, in addition to an insulating material, e.g., air, the one or more lumen contain cuts. The multi-lumen outer sleeve surrounds the thermal medium 235 and as shown in FIG. 2b, the components may be manufactured as an integral unit, of identical or dissimilar materials, using known fabrication techniques such as co-extrusion or molding. Within the thermal medium one or more heating elements 238 are provided to surround a fluid delivery tube 242. In this embodiment, the fluid delivery tube component is also manufactured integral to the thermal medium and hence outer sleeve. The fluid delivery tube includes a sterile fluid pathway 245 for fluid which are warmed therein.

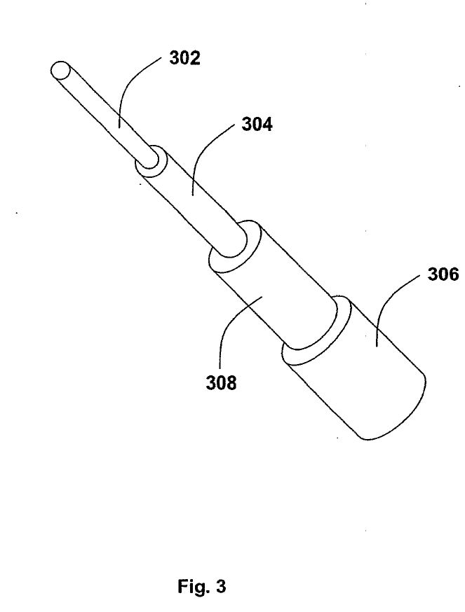

The heating element may include a flexible heat-tape, such as, for example, either series or parallel resistance heaters. As shown in FIG. 3, such heating elements generally include one or more wires 302 that produce heat upon an electrical current running through the wire. The wire(s) may be enveloped in a semi-conductive matrix 304, which may be further enveloped by an insulative material 308. An outer-jacket 306 may also be included.

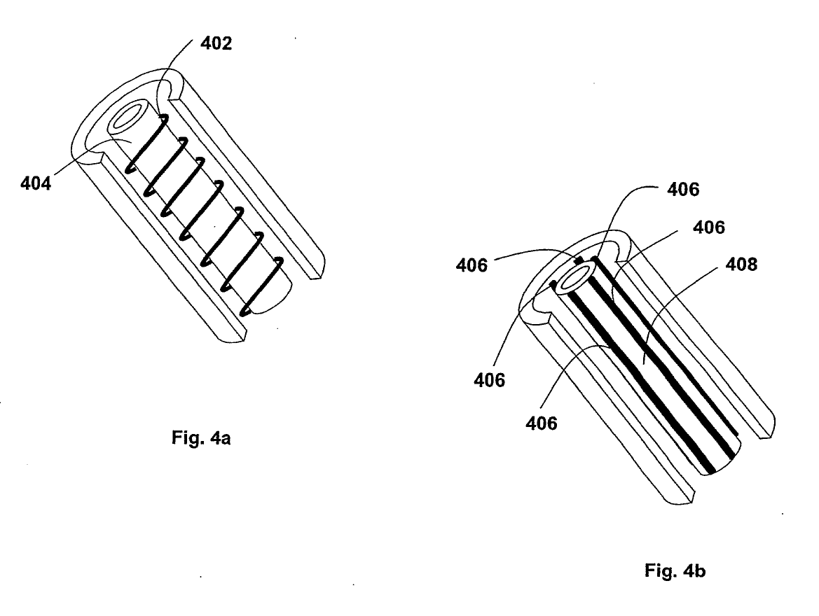

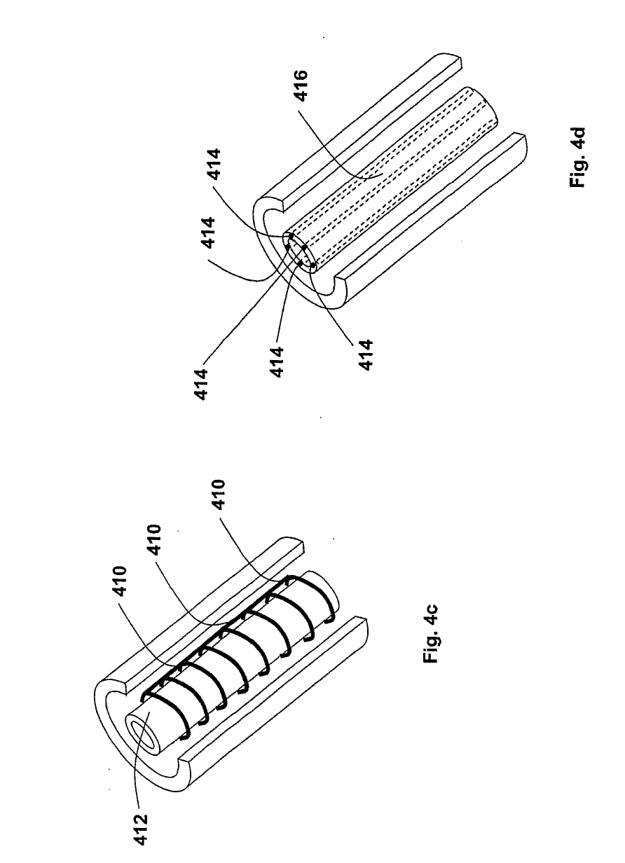

As shown in FIGS. 4A-4D, the heating element(s) may be arranged in a number of ways. FIG. 4A illustrates the use of a coiled heating element 402, which may be spirally wound around the inner fluid delivery tube 404. In another embodiment of the invention, the wire pitch of the coiled heating element is from about 0.1 to about 0.5. In another embodiment of the invention, the wire pitch of the coiled heating element is from about 0.1 to about 0.4. In another embodiment of the invention, the wire pitch of the coiled heating element is from about 0.17 to about 0.33. In another embodiment, two or more wire heating elements are spirally wound around the inner fluid delivery tube. In another embodiment of the invention, the two or more wire heating elements are connected in parallel. As shown in FIG. 4B, the heating element may include several heating elements 406 positioned linearly along the length of the inner fluid delivery tube 408. In one embodiment of the invention, the heater wire maintains at least about 0.06″ between the heater wire and the fluid. This ensures an appropriate resistance to current leakage. In one embodiment of the invention, the heater wire maintains at least about 0.06″ to about 0.5″ between the heater wire and the fluid. In another embodiment of the invention, the heater wire maintains at least about 0.06″ to about 0.25″ between the heater wire and the fluid. In one embodiment if the invention the tubing has an ID at least about 0.05″. In another embodiment of the invention, the tubing has an ID of from about 0.1″ to about 0.5″. In another embodiment of the invention, the tubing has an ID of about 0.1″ to about 0.3″. FIG. 4C illustrates the uses of a plurality of interconnected heating elements 410 placed along the length of the inner fluid delivery tube 412. FIG. 4D illustrates the use of several heating elements 414 placed within the wall of the inner fluid delivery tube 416. The heater wire is embedded in the extrusion and may be of any orientation, e.g., but not limited to, straight or wrap. In another embodiment of the invention, there are from about two to about twenty heater wires in the tubing. In another embodiment of the invention, there are from about two to about fifteen heater wires in the tubing. In another embodiment of the invention, there are from about four to about twelve heater wires in the tubing. In such an embodiment, the heating element may only include the one or more wires or the one or more wires with the semi-conductive matrix and/or insulative material (see FIG. 3).

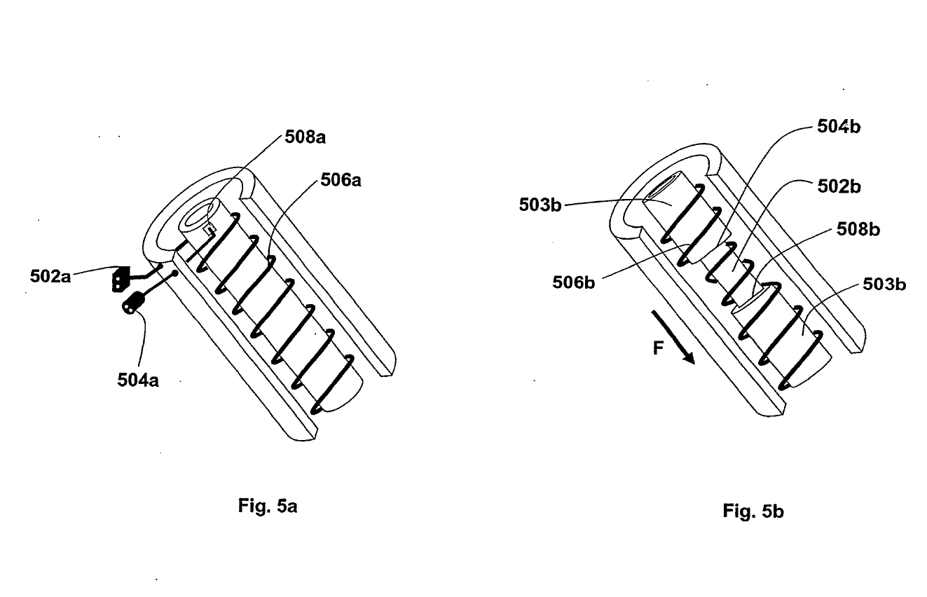

As shown in FIG. 5A, connectors 502a and 504a, are provided on the fluid delivery-line for connecting the heating element 506a and the thermal sensor 508a, to corresponding connections on the controller 104. The connectors may be formed into one connector, where electrical connections for each are formed therein to connect to the controller. Accordingly, the controller connection may include one connector having electrical connections for the heating element and the thermal sensor, or two separate connectors.

In some embodiments of the present invention, the connector that provides electrical current from the power source to the fluid delivery-line heater element, is incorporated within a multi-function tube fitment that is assembled with the fluid delivery-line at the time of manufacture. The multi-function fitment also attaches or docks the fluid delivery-line to a fluid container, additional tubing, or the patient, via integral hose barb, luer, and/or other IV fluid connections. Additionally, the fitment may contain one or more ports for the insertion thermal or other type sensors. The described sensor ports provide either direct contact with the fluid stream or access to a contained location proximal to or surrounded by the fluid stream. The fitment may include a cover or protective wrap component.

In some embodiments of the present invention, as shown in FIG. 5B, one or more sections of a heat conductive material 502b, for example a metallic material (e.g., stainless steel) is provided along the fluid delivery tube 503b to enhance heat flow. In some embodiments of the present invention, the heat conductive material includes a first portion 504b (e.g., an end portion) in contact with one end of the fluid delivery tube 503b, and a second portion 608b (e.g., the other end portion) in contact with the other adjacent end of the fluid delivery tube concentric the fluid flow F making contact therewith. Thus, the heat generated by the heating element moves from the first portion to the second portion of the heat conductive material to pass a higher amount of heat to the fluid within the fluid delivery tube. Preferably, the heat conductive material is positioned at the bag-spike end of the fluid delivery-line or closer to the bag-spike end than the end having the luer-lock and/or transfusion needle, so that heat variations, if any, along the fluid delivery-line are eliminated or substantially reduced by the time the fluid arrives at the transfusion needle.

The heat conductive material may be of any shape or form, which enables one portion to be exposed to the heat generated by the heating element and another portion to be exposed to the fluid within the tube. Thus, rod shapes, flat sheets, coils, and the like, may be used.

These types of embodiments may be used for specialized applications, for example, requiring a shorter tube length or higher flow-rate, or a combination thereof, than a normal application. Such specialized applications include hypothermia related injuries.

One of ordinary skill in the art will appreciate that the one or more heating elements may be interconnected and may be placed next to the outer surface of the inner fluid delivery tube, or may be spaced apart from the outer surface of the inner fluid delivery tube. In that regard, the one or more heating elements may be placed within the thermal medium, between the inner surface of the outer sleeve of insulation and the outer surface of the inner fluid delivery tube.

The one or more thermal sensors may be thermisters, which are thermally sensitive resistors, which are solid state, electronic devices for detecting thermal environmental changes. In one embodiment, the one or more thermal sensors may be positioned at the end of the fluid delivery tube near the transfusion needle. In such an embodiment, the valve 112 may be positioned near the transfusion needle to control the flow of fluid from the fluid delivery-line into the patient. Accordingly, the temperature of the fluid within the fluid delivery tube may control the valve. When the temperature of the fluid within the fluid delivery tube reaches a predetermined temperature (i.e., after the heating element provides heat to the fluid delivery tube), the valve opens and allows the fluid to flow.

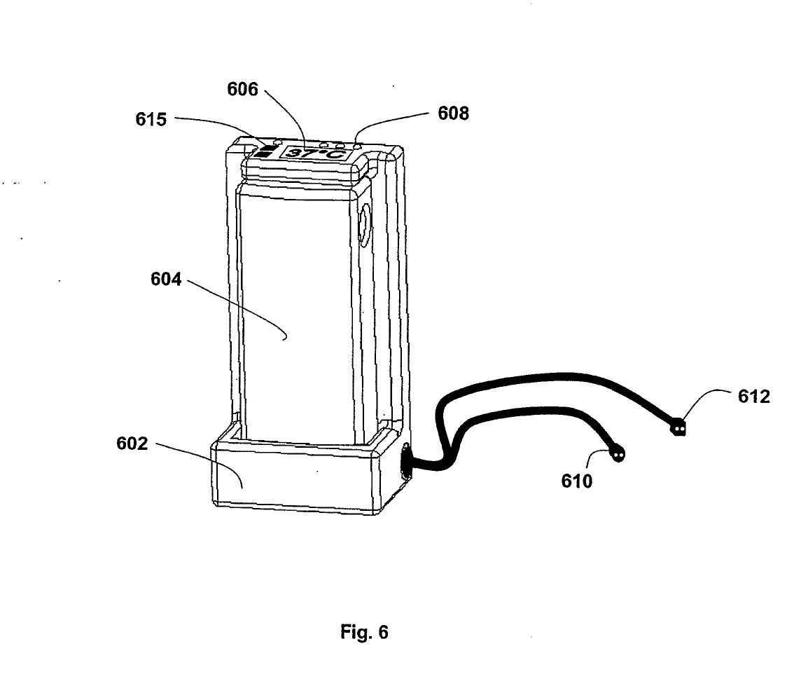

The controller, as shown in FIG. 6, may include a housing 602 made of plastic or other similar material, which houses the circuitry for providing the electrical current and sensing the temperature of the fluid within the fluid delivery tube. The controller may also include a battery pack 604 or other power source (external or internal), a temperature display 606 for indicating a temperature of the fluid within the inner fluid delivery tube, and one or more LED lights 608. The LEDs may be used to indicate any one of the following: power level of the power source, whether the controller is connected to the heating element and/or thermal sensor, indicator light for a temperature within a prescribed range (e.g., for delivery to a patient, too hot and/or too cold). The controller may also include a speaker for audio signals.

Connectors 610 and 612 connect the controller to the corresponding connectors for the heating element(s) and thermal sensor(s) of the fluid delivery-line. These connectors may include a locking feature that insures that connections do not come apart and/or that the connectors are fully connected.

The controllers of the warmer unit and warming cabinet may be implemented by any quantity of any conventional or other microprocessor, controller or circuitry, and may each control any quantity of compartments. The warmer unit and warming cabinet may include any quantity of any types of displays (e.g., LCD or LED) of any shape or size and disposed at any locations on or remote from the warmer unit and warming cabinet. The controls may be of any quantity, shape or size, maybe implemented by any suitable input devices (e.g., keypad, buttons, voice recognition, etc.) and may be disposed at any locations on the warmer unit and warming cabinet. The warmer unit and warming cabinet displays may each be associated with and provide information for any quantity of receptacles and may include any quantity of display fields including any desired information. Further, a display may selectively provide any information (e.g., residence time, insertion time, desired and actual temperatures or other information individually or in any combinations) for each receptacle or for any portion of the total quantity of receptacles. The display may be updated periodically, at any desired time interval and/or in response to the counters, controller input devices, controls and/or any desired conditions. A display field may correspond to and provide information for any quantity of receptacles, while the fields and receptacles may be associated by any type of identifier (e.g., alphanumeric identifier, symbols, icons, etc.). The display may alternatively provide any desired information in any format to a user. The warmer unit and warming cabinet may provide any visual (e.g., flash, bold, identify receptacle, etc.) and/or audio (e.g., beep or other sound, synthesized speech, etc.) alarms to notify a user of any desired conditions (e.g., item attaining or exceeding the set point or other temperature, time limit exceeded, etc.).

The controller may receive a compartment temperature and individual set point temperatures for each item. Thus, items associated with different set point temperatures may be heated within the same compartment, while the system notifies the user when each item has attained or exceeded the corresponding set point temperature via the visual and/or audio alarm. The counters may be implemented by any hardware (e.g., registers, circuitry, etc.) or software and may be incremented in response to any time interval (e.g., controller system clock, seconds or any fractions thereof, etc.) and/or conditions.

The controller may include any quantity of any types of displays (e.g., LCD, LED, etc.) of any shape or size and/or any quantity of any type of input devices (e.g., keypad, buttons, etc.) of any shape or size. The display and input devices may be disposed at any suitable locations on the controller and facilitate display and entry of any desired information.

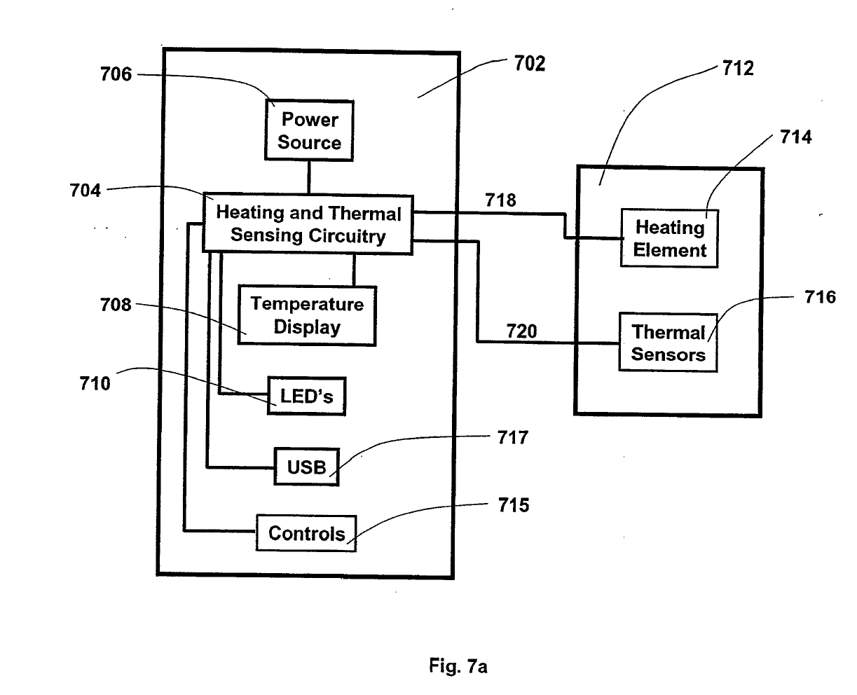

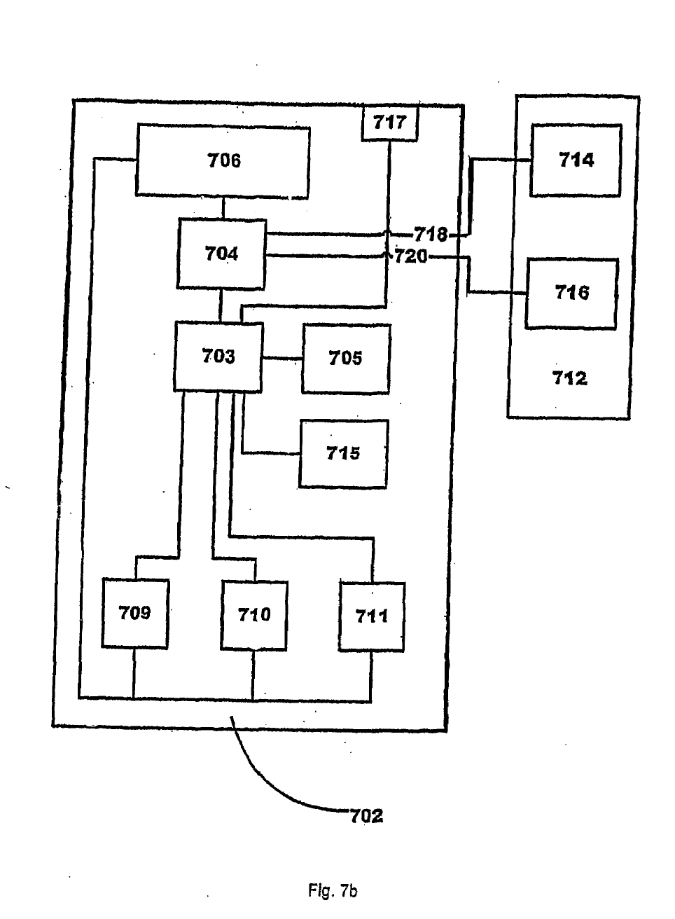

Schematic diagram illustrating two embodiments of the controller 702 are shown in FIG. 7A and FIG. 7B, respectively. One of skill in the art will appreciate that the one or more of the various circuits/circuitry of the controller of some of the embodiments of the present invention may be analog or digital.

As illustrated in FIG. 7A, in one embodiment, upon the controller including digital circuitry, for example, the controller 702 may include a heating and/or thermal sensing circuitry 704. A power source 706 may also be provided internal or external to the controller. A temperature display 708, LED circuitry 710, a communication port, e.g., USB port 717, and controls 715 may also be provided. The heating and sensing circuitry 704 may be connected to the heating element(s) 714 and thermal sensor(s) 716 of the fluid delivery-line 712 via connections 718 and 720, respectively.

As illustrated in FIG. 7B, in another embodiment, upon the controller including digital circuitry, for example, the controller may include a microprocessor 703, having memory 705 (which may be a detachable memory module), which communicates to heating and/or thermal sensing circuitry 704. A power source 706 may also be provided internal or external to the controller. LED circuitry and/or display 709, audio circuitry and/or output 710 and a temperature circuitry and/or display 711 may also be provided, each of which may communicate with the microprocessor. The heating and sensing circuitry 704 may be connected to the heating element(s) 714 and thermal sensor(s) 716 via connections 718 and 720, respectively.

Controls 715 may also be included which may be used to set them temperature for the fluid (to be heated to, for example), or for setting different parameters of the controller. For example, the memory may include heating routines for a specific type of fluid. Using controls 715, a user can then select an appropriate heating routine.

A serial port or USB port 717, for example (which may be any type of communication port familiar to one of skill in the art), may be included which allows the controller to communicate with a computer. Such communication may then be used to perform calibration tests, for example, and download heating information for heating particular types of fluids.

The temperature display may be used to display a visual indicator of the temperature, e.g., an actual digital display of the temperature of the fluid. The LEDs may be used to monitor the temperature as well, and may also be used to indicate certain conditions of the controller and/or fluid delivery-line. For example, the LEDs may indicate that the controller is on or off, that the temperature of the fluid has reached a predetermined value, that current is being sent to the heating element, and the like. The audio circuitry/output may be used to provide audio indication that fluid has reached a desired temperature, for example.

The controller may also include digital/analog conversion circuits for operating the heating element and collecting temperature information from the one or more thermal sensors. Moreover, in some embodiments of the present invention, one or more (or all) functions of the controller may be replaced by a computer (desktop, mini/micro, mainframe, PDA and the like), having connectors and corresponding circuitry to carry out the application and control of current to the heating element, the sensing of temperature, and/or the actuation of a valve for controlling the flow of fluid through the fluid delivery-line of the present invention.

The controller may include other features such as a variable temperature selector for changing a resultant temperature of the fluid within the inner fluid delivery-line 615. Thus, if, for example, a patient is suffering from hypothermia, a medicating fluid (e.g., to aid in the recovery of the patient) may be kept at a temperature above the body temperature of the patient, but below normal. Accordingly, the heating and thermal sensing circuitry may include circuitry for gradually increasing a resultant temperature of the fluid within the fluid delivery tube to aid the recovery of a hypothermia patient. In that regard, the heating and thermal sensing circuitry may include circuitry for gradual increase or decrease of a resultant temperature of the fluid within the fluid delivery tube for any number of therapeutic reasons. Of course, a range of temperatures within which the controller and present system may operate may be, e.g., between 32° F. and 105° F.

The controller may also include circuitry for actuating valve 112. Such circuitry may be integral or connected to the heating and thermal sensing circuitry such that upon the thermal sensing circuitry detecting the resultant temperature of the fluid within the inner fluid delivery tube being at a predetermined temperature, the circuitry actuates the valve to allow the fluid to flow into the patient. Accordingly, the circuitry may be connected to the valve via a wire, which sends current to an electro-mechanical actuator at the valve.

In some circumstances, patients may require pre or post-operative cooling for a variety of reasons, including, for example, treatment of a malignant hypothermia crisis and induction of therapeutic hypothermia for neurosurgery.

It is within the scope of the present invention that the system of the present invention can be used for cooling a fluid. In one embodiment, the heat element is replaced with a hollow tube for circulating a coolant or a solid metallic chilling element that serves to lower the temperature of the fluid in the delivery-line. This configuration may be used in the delivery of cooled fluid to a patient, for I.V. use and/or other fluid administration techniques.

In another aspect of the present invention, the system has both heating and cooling elements and can be used for warming and cooling, thereby controlling the temperature of a fluid, the temperature of a target tissue, or the temperature of a patient.

The present system may be used with any types of power sources, e.g., AC, DC, wall outlet jack, batteries, vehicle power systems. The present system may be mounted on, or supported by, any type of support structure, e.g., wall, cart, table, floor. The systems preferably heat or cool items to desired temperatures within the approximate range of 70° F. to 150° F.

### Embodiments of Select Components of the Fluid Warmer

Design options useful for the fluid warmer of the invention can improve the function of the fluid warmer of the present invention in different applications and the temperature sensing capability, as well as lower cost of manufacturing the components, e.g., delivery-line component.

A. The Delivery-Line Component of the Invention

In one embodiment of the invention, the fluid delivery-line component 102 is made of silicone. This material can act as fluid tube, heat distribution tube or insulations tube. In another embodiment of the invention the fluid delivery-line component is made of medical grade silicone. An example of medical grade silicone is Class VI silicone. In one embodiment of the invention, the extruded silicone thickness is from about 0.5 Watts/inch to about 7.5 Watts/inch. In another embodiment of the invention, the extruded silicone thickness is from about 2 to about 5 Watts/inch. In another embodiment the pitch of the wire is altered.

Silicone is useful as a material in the invention because it is a pure material that does not leak chemical components into the system of the invention, e.g., plasticizers or oxidants. The contamination of the fluids within the system of the invention by such leakage from the fluid delivery-line component material is not desirable because the fluid may be delivered to a subject. Silicone is also useful in the fluid delivery-line component of the present invention because it has heat insulation property that aids in a uniform distribution of heat within the material. The more uniform distribution of heat provided by silicone is advantageous because it prevents the formation of “hot-spots” that damage heat-sensitive components of fluids such as found in, e.g., blood. Further, silicone is advantageous for use in the fluid delivery-line component of the present invention because of the low heat capacity of this material. The low heat capacity of silicone reduces the lag-time between a reduction of the temperature setting of the system and a commensurate reduction of heating of the fluid in the system. Residual heating of fluid in the system of the present invention due to lag-time is not advantageous because the control of the fluid temperature is not optimal and fluid continues to be heated even after the heating element has been turned off. Another advantage of the use of silicone fluid delivery-line component is the high heat current leakage resistance of this material. The high current leakage resistance of the silicone prevents electrocution of a subject in contact with the system of the invention. In one embodiment of the invention, the inner wall thickness of fluid delivery-line component is maintained at least about 0.06″ (i.e., 0.06 inches). Maintaining the inner wall thickness of silicone of at least about 0.06″ is advantageous because it aids in preventing current leakage and subsequent electrocution of a subject in contact with the system of the invention. In another embodiment of the invention, the outer wall thickness of fluid delivery-line component is varied.

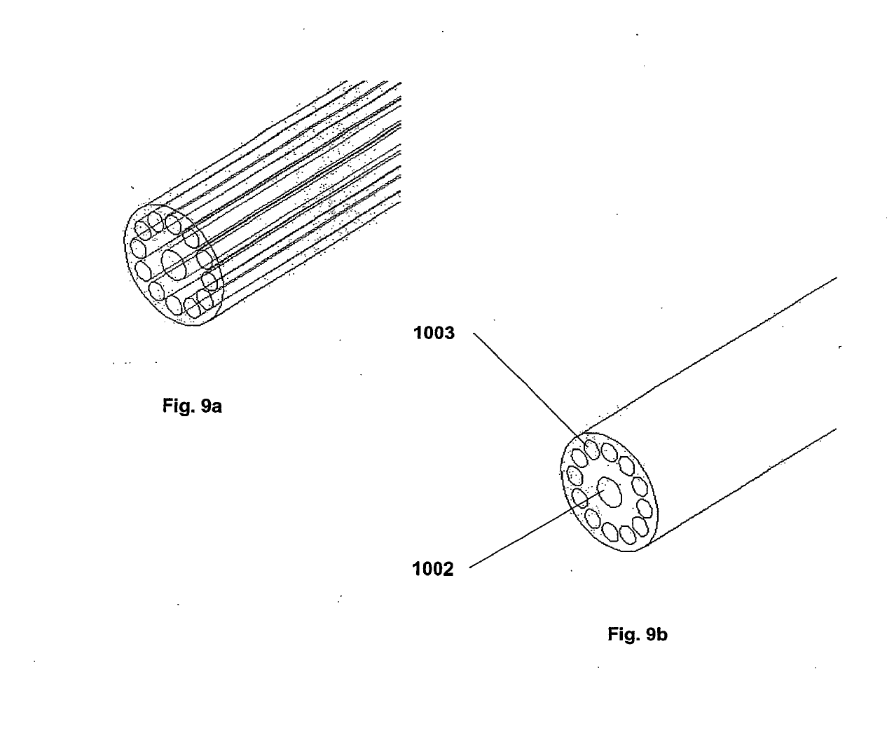

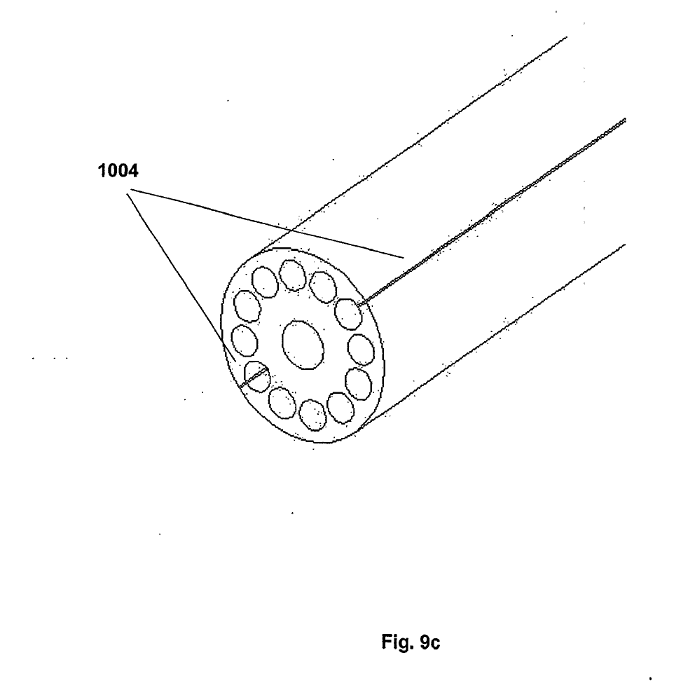

As shown in FIG. 9A and FIG. 9B, in one embodiment of the invention, the fluid delivery-line component of the invention has an inner lumen 1002 and an outer lumen 1003. In one embodiment of the invention, the fluid delivery-line component of the invention has from about two to about twenty outer lumen. In one embodiment of the invention, the fluid delivery-line component has from about two to about fifteen outer lumen. In one embodiment of the invention, the fluid delivery-line component has from about five to about fifteen outer lumen. In another embodiment of the invention the fluid delivery-line component has twelve outer lumen. In one embodiment of the invention, fluid is circulated in the outer lumen of the fluid delivery-line component 1003. In another embodiment of the invention, fluid is circulated in the inner lumen of the fluid delivery-line component 1002. Circulation of fluid in the lumen of the fluid delivery-line component is useful to cool, heat or insulate.

In one embodiment of the invention, the outer lumen of the fluid delivery-line component is used as a conduit. In one embodiment of the invention, the outer lumen of the invention is used as conduit for wire. The wire can be wire for different purposes, e.g., heater power supply wire or temperature sensor wire.

As shown in FIG. 9C, in one embodiment of the invention, an outer lumen of the fluid delivery-line component is pierced. In one embodiment of the invention, an outer lumen is pierced as a slit along a length of the fluid delivery-line component. Piercing an outer lumen can allow for access to the outer lumen for, e.g., placement of a wire (e.g., heater supply wire or temperature sensor wire). The fluid delivery-line component can be pierced at the time of extrusion or after extrusion of the fluid delivery-line component. The piercing can be later re-sealed with RTV adhesive or covered with a thin film of polyolefin, or the like.

The diameter of the fluid delivery-line component and the diameter of the outer lumen can be varied. This allows for removal of material to lower the cost of manufacture while maintaining a set distance from the heater wire to the contact area (e.g., O.D.). In one embodiment of the invention, fluid delivery-line component of the invention is from about 0.1″ to about 1″ O.D. In another embodiment of the invention, the fluid delivery-fine component is from about 0.25″ O.D. to about 0.75″ O.D. In another embodiment of the invention, the fluid delivery-line component is about 0.5″ O.D. In one embodiment of the invention, the outer lumen of the fluid delivery-line component is from about 0.01″ to about 0.2″. In another embodiment of the invention, the outer lumen of the fluid delivery-line component is from about 0.05″ to about 0.15″. In yet another embodiment of the invention, the outer lumen of the fluid delivery-line component is about 0.08″. In one embodiment of the invention, the bolt diameter circle of the invention is from about 0.1″ to about 1″. In anther embodiment of the invention, the bold diameter circle is from about 0.2″ to about 0.7″. In yet another embodiment of the invention, the bolt diameter circle is from about 0.3″ to about 0.4″. In yet another embodiment of the invention, the bolt diameter circle is about 0.36″.

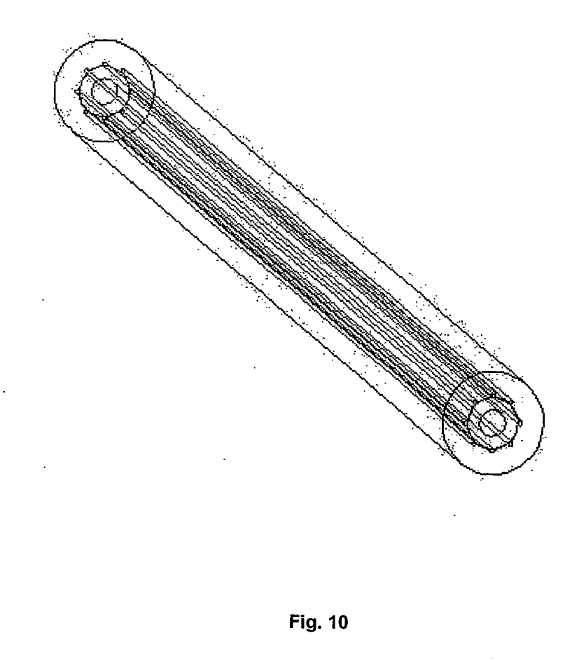

As shown in FIG. 10, in one embodiment of the invention, the fluid delivery-line component of the invention has one or more heater wires in the fluid delivery-line component. In another embodiment of the invention, the heater wire is straight. In another embodiment of the invention, the wire is spiral wrap around the lumen of the fluid delivery-line component. In one embodiment of the invention, the heater wire is spiral wrap at a rate of at least about one wrap per foot. In another embodiment of the invention, there are from about two to about twenty heater wires in the fluid delivery-line component. In another embodiment of the invention, there are from about ten to about twenty heater wires in the fluid delivery-line component. In another embodiment of the invention, there are from about four to about eight heater wires in the fluid delivery-line component. In another embodiment of the invention, the heater wires are connected together using an end fitment. In yet another embodiment the end of the wire is flush to the surface of the fluid delivery-line component. In yet another embodiment the end of the wire extends beyond the surface of the fluid delivery-line component. Extension of the end of the wire beyond the surface of the fluid delivery-line component exposes the end of the wire for easy access and connection.

The wire gauge and type can be altered to suit a variety of processes. In one embodiment of the invention, the heater wires of the invention can be of a wire gauge and material(s) that are better for manufacturing. In another embodiment of the invention, the wire pitch is from about 0.1 to about 0.5. In another embodiment of the invention, the wire pitch is from about 0.1 to about 0.4. In another embodiment of the invention, the wire pitch is from about 0.17 to about 0.33. The electronics of the invention can handle a wide array of loads and the watt density can also be varied.

In one embodiment of the invention, the pitch of the wire is decreased to alter the run rate. In another embodiment the pitch is increased to alter the run rate. In yet another embodiment of the invention, the pitch is decreased such that the run rate is increased.

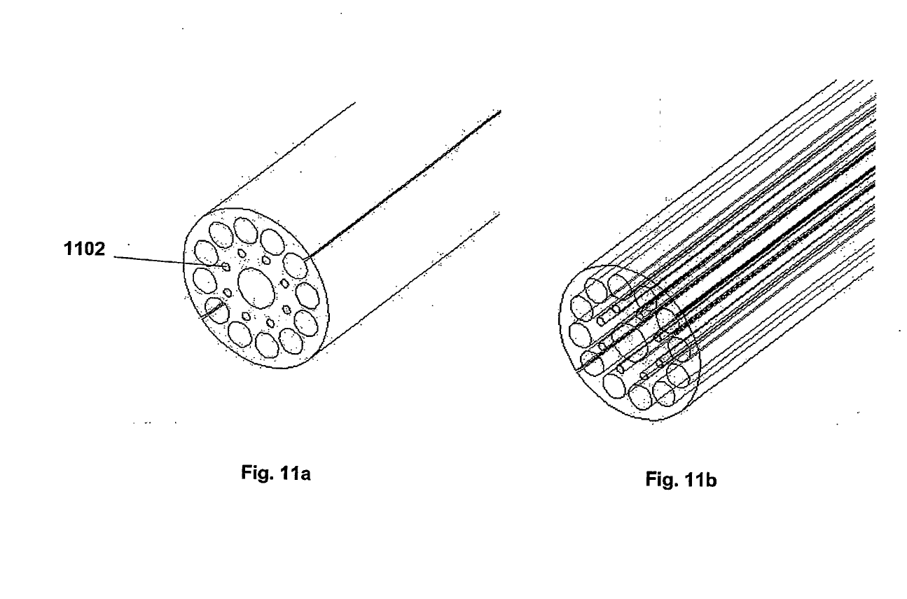

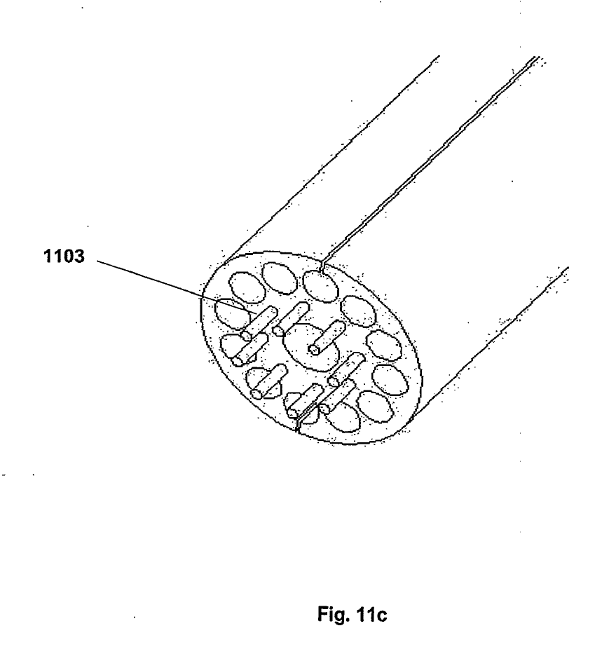

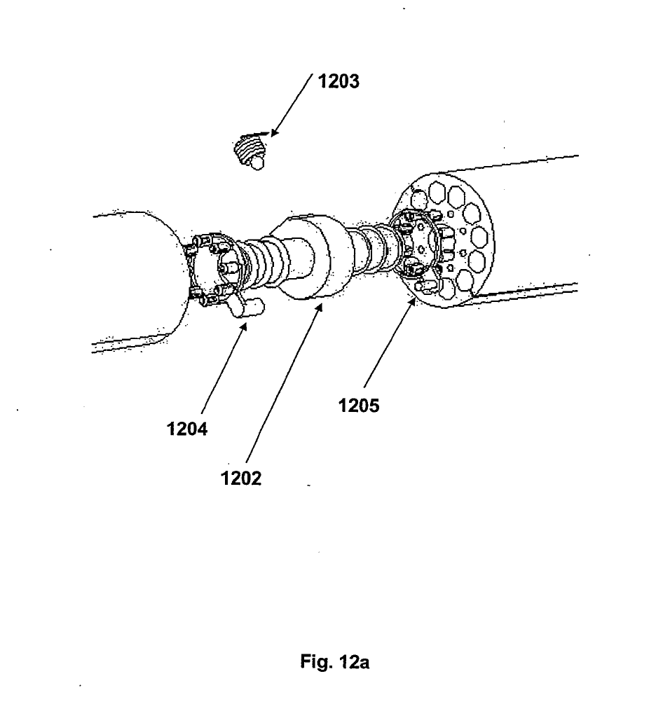

Another embodiment of the invention is illustrated in FIG. 11. As shown in FIG. 11, the features of the design described above in FIG. 9 and the features of the design described above in FIG. 10 can be combined. The features of the combined fluid delivery-line component design of FIG. 11 can be varied as detailed above in FIG. 9 and FIG. 10. As shown in FIG. 11A and FIG. 11B, in one embodiment of the invention, the end of the wire is flush to the surface of the fluid delivery-line component 1102. As shown in FIG. 11C, in another embodiment of the invention, the end of the heater wire extends beyond the surface of the fluid delivery-line component 1103.

B. Fitments of the Invention

The invention provides for mid-fitments (a.k.a, union fitting or union fitment) and end-fitments. A mid-fitment connects two lengths of fluid delivery-line component within the system of the invention. An end-fitment is placed on one end of a length of fluid delivery-line component in the system of the invention. The fitments of the invention can be made of any suitable material. In one embodiment of the invention, the fitments are injection molded or LIM. In other embodiments of the invention, the fitments are made of PVC or silicone, e.g., high durometer silicone. In one embodiment of the invention the fitments has barb-type fittings for connection to fluid delivery-line component. The fitments may be further secured using a suitable adhesive to increase the strength of the connection between the fitment and the fluid delivery-line component. Adhesive is useful in applications where higher pressures are created within the system, e.g., trauma application.

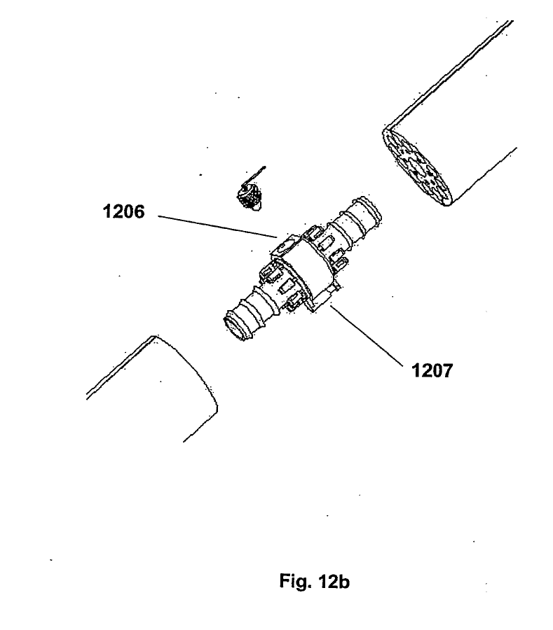

A mid-fitment assembly is illustrated in FIG. 12. As shown in FIG. 12A, in one embodiment of the invention, a mid-fitment 1202 is used to connect two lengths of fluid delivery-line component in the system. More than one mid-fitment can be placed within the system of the invention. Mid-fitments can be place anywhere along the length of the fluid delivery-line component 102 of the system. In one embodiment, a female connector 1204 is placed on one end of the mid-fitment. In another embodiment of the invention, a male connector 1205 is placed on one end of the mid-fitment. The female connector 1204 and the male connector 1205 are useful to connect wires in the fluid delivery-line component such that they connected to one another or can be accessed for connection to other components of the system of the invention, e.g., a power supply or lead. In one embodiment of the invention, a sensor-mounted gasket 1203 is placed in the mid-fitment. The sensor is a temperature sensor that is placed in contact with the fluid such that there is direct sensing of the temperature of the fluid in the system. As shown in FIG. 12B, well 1206 is located in the mid-fitment to receive the gasket and temperature sensor. In one embodiment of the invention, the temperature sensor is placed in the mid-fitment without the use of a gasket. The temperature sensor can be secured in the mid-fitment with any suitable material. Also shown in FIG. 12B, is the interconnection of a female connector and male connector which, in turn, connect heater wires in the fluid delivery-line component upon full assembly of the mid-fitment assembly 1207.

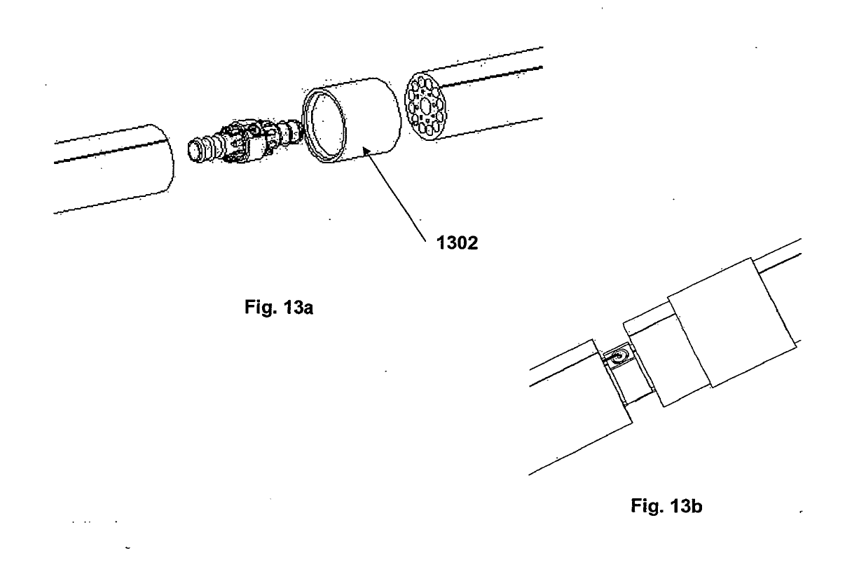

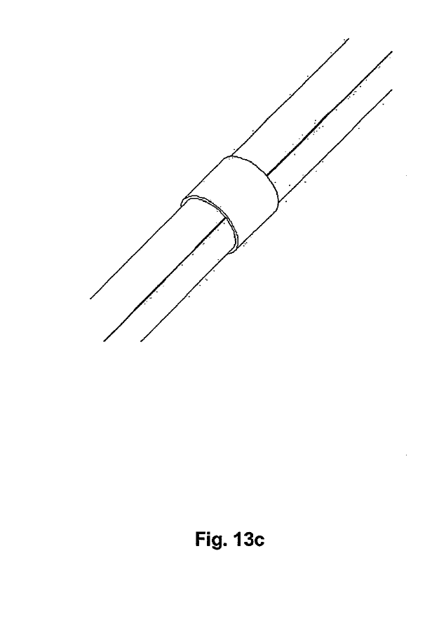

As shown in FIG. 13A, in one embodiment of the invention, a collar 1302 is positioned over the end of the mid-fitment. As shown in FIG. 13B, the collar has an internal diameter sufficient to fit over the fluid delivery-line component. As shown in FIG. 13C, the collar is placed over the mid-fitment assembly. In one embodiment of the invention the collar is an interference fit. In another embodiment of the invention, the collar is adhered in place. The collar assists in securing the mid-fitment assembly and is useful to protect the components of the mid-fitment assembly from disruption, e.g., mechanical disruption or moisture. The collar provides an added physical barrier to maintain the sterility of the system. The collar also protects a subject from coming into contact with the components of the mid-fitment assembly leading to disruption of the mid-fitment assembly or potential to electrocution of a subject.

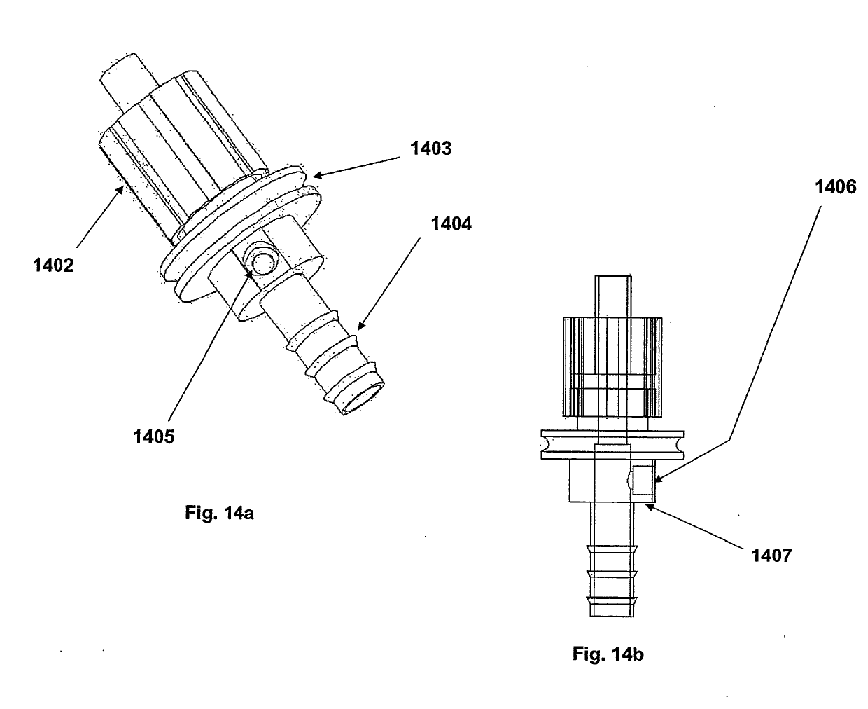

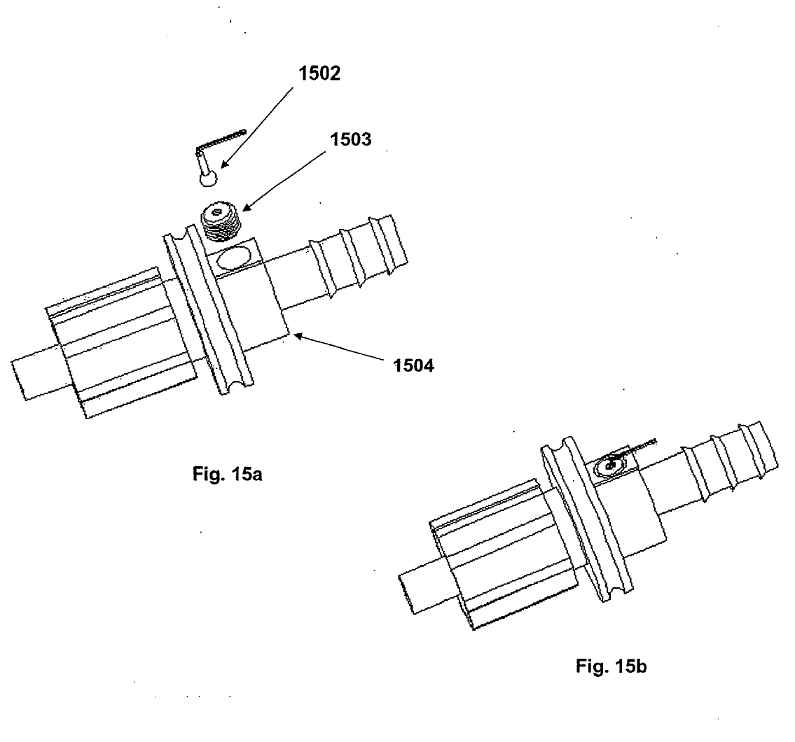

An end-fitment of the invention is illustrated in FIG. 14. As shown in FIG. 14A, in another embodiment of the invention, the fitment is an end-fitment. The end-fitment is useful to connect an end of a fluid delivery-line component of the system to a terminal fitment (e.g., a needle or catheter), or to connect an end of a fluid delivery-line component of the system to a fitment connected to a source bag. As shown in FIG. 14A, the end-fitment of the invention has a luer-lock feature 1402 that secures another fitment, e.g., a needle, catheter or fitment connected to a source bag. The end-fitment also has a collar-lock feature 1403 to secure a fitted collar over the end-lock assembly. In one embodiment of the invention, the end-fitment has a temperature sensor well feature (See FIG. 14A, feature 1405; FIG. 14B, feature 1406). In one embodiment of the invention, a sensor-mounted gasket is placed in the end-fitment. The sensor is a temperature sensor that is placed in contact with the fluid such that there is direct sensing of the temperature of the fluid in the system. As shown in FIG. 14A and FIG. 14B, well 1405 and 1406 is located in the end-fitment to receive the gasket and temperature sensor. In one embodiment of the invention, the temperature sensor is placed in the end-fitment without the use of a gasket. An adhesive material, e.g., silicone (e.g., RTV) or epoxy, can be dispensed in the temperature sensor well to secure the temperature sensor in the end-fitment. The end-fitment of the invention also has a shelf 1407 for a heater element push-lock connector or with center ring cut-out for crimp and solder-style heater element connector clearance.

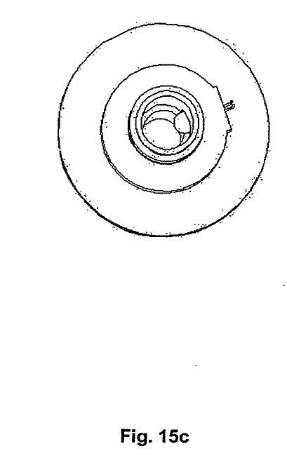

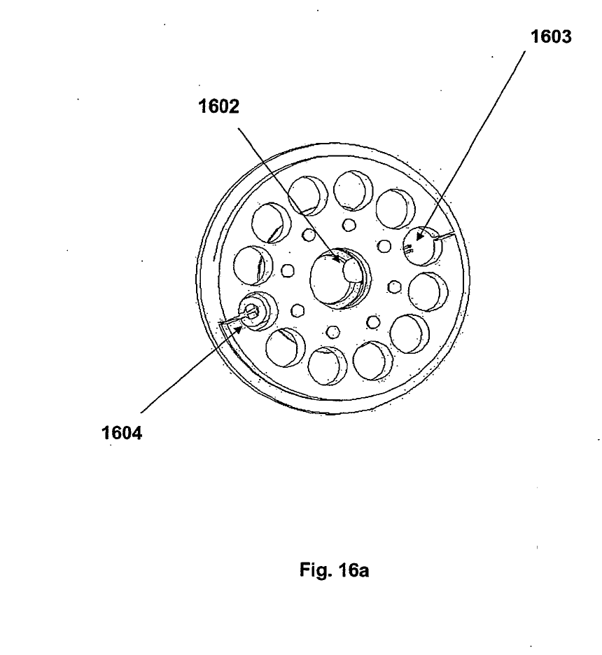

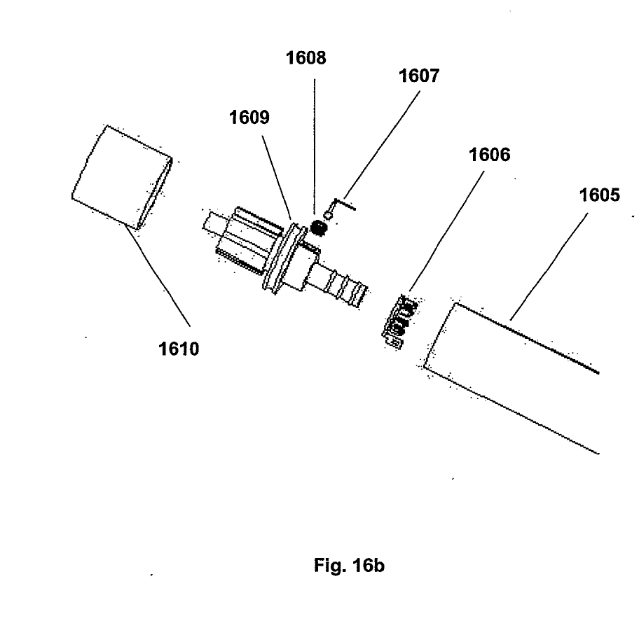

FIG. 15A further illustrates the temperature sensor 1502, sensor gasket 1503 and end-fitment 1504 useful in some embodiments of the end-fitment assembly of the invention. FIG. 15B illustrates the placement of the temperature sensor and sensor gasket in the sensor-well of the end-fitment. FIG. 15C illustrates a view of the end-fitment illustrating the exposure of the temperature sensor to inner lumen such that it contact the fluid for direct temperature sensing measurement. Similarly, FIG. 16A illustrates the exposure of the temperature sensor 1602 to the inner lumen such that it contacts the fluid for direct temperature sensing measurement. FIG. 16A further illustrates the alignment of the temperature sensor leads with the extrusion lumen in order for the lumen to act as a wire-way 1603. This is particularly relevant where the fluid delivery-line component has outer lumen used a conduit for the temperature sensor wire. The location of the heater element wire is also notable 1604.

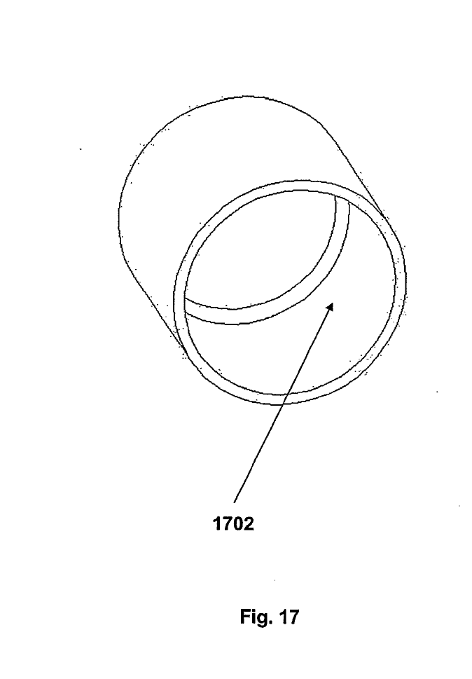

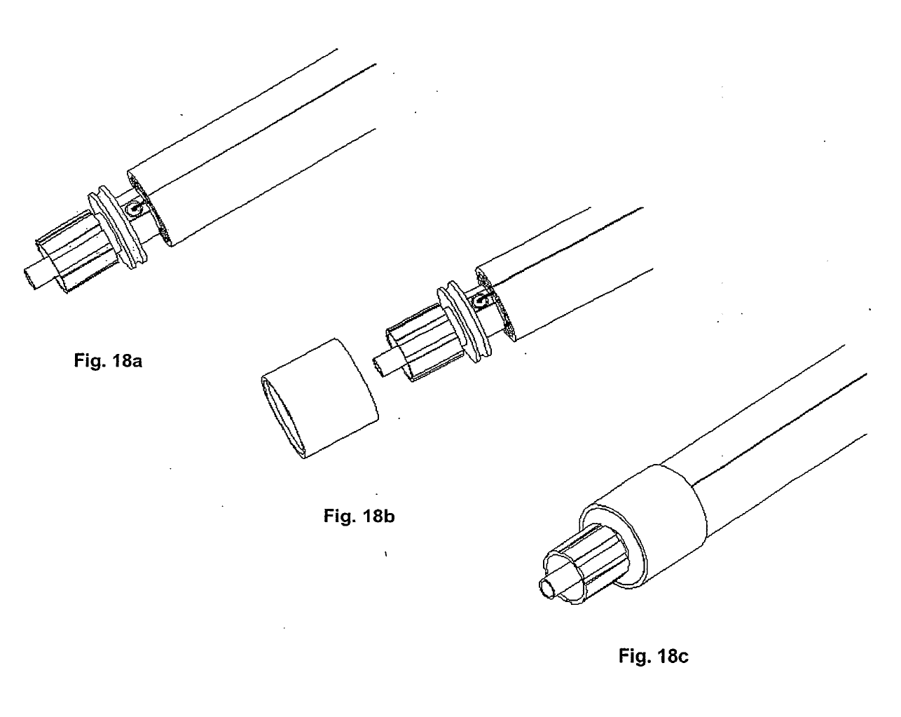

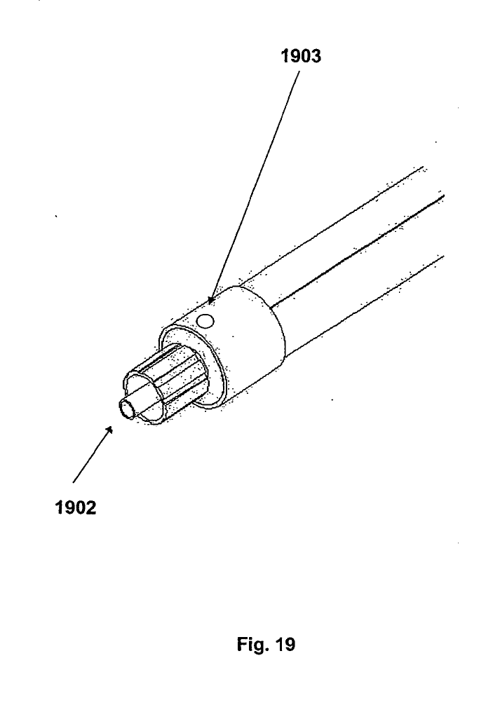

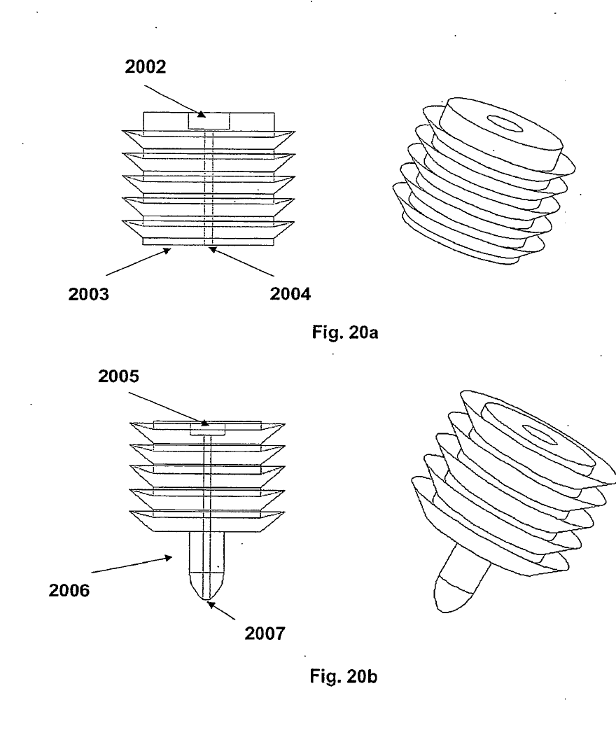

An end-fitment assembly (e.g., general assembly of end of warmer disposable set) is illustrated in FIG. 16B. In one embodiment of the invention, a connector 1606 is placed on one end of the end-fitment 1609. The connector 1606 is useful to connect wires in the fluid delivery-line component 1605 such that they connected to one another or can be accessed for connection to other components of the system of the invention, e.g., a power supply or lead. In one embodiment of the invention, a sensor-mounted gasket (1607 and 1608) is placed in the end-fitment. The sensor is a temperature sensor 1607 that is placed in contact with the fluid such that there is direct sensing of the temperature of the fluid in the system. As shown in FIG. 16B, in one embodiment of the invention, a collar 1610 is positioned over the end of the end-fitment. As shown in FIG. 16B, the collar has an internal diameter sufficient to fit over the fluid delivery-line component and the collar is placed over the end-fitment assembly. As shown in FIG. 17, in one embodiment of the invention, the collar has a mating-lock feature 1702 useful for connecting to the leur fitment. In one embodiment of the invention, the collar is an interference fit. In another embodiment of the invention, the collar is adhered in place. The collar assists in securing the end-fitment assembly and is useful to protect the components of the end-fitment assembly from disruption, e.g., mechanical disruption or moisture. The collar acts as an added physical barrier to maintain the sterility of the system. The collar protects a subject from coming into contact with the components of the end-fitment assembly leading to disruption of the end-fitment assembly or potential to electrocution of the subject. The collar also squeezes the silicone fluid delivery-line component to secure the leur fitment to the fluid delivery-line component. FIG. 18 further illustrates an end-fitment assembly. As shown in FIG. 18A, the end-fitment with temperature sensor is placed into the end of the fluid delivery-line component. As shown in FIG. 18B and FIG. 18C, the collar is fitted over the end-fitment to cover the temperature sensor. Another embodiment of the present invention is shown in FIG. 19. In this embodiment of the present invention, the collar on the end-fitment assembly has an orifice. The orifice in the collar is useful to act as an exit point for a wire(s) in the fluid delivery-line component. The orifice can be easily sealed.

C. Temperature Sensor Gaskets of the Invention

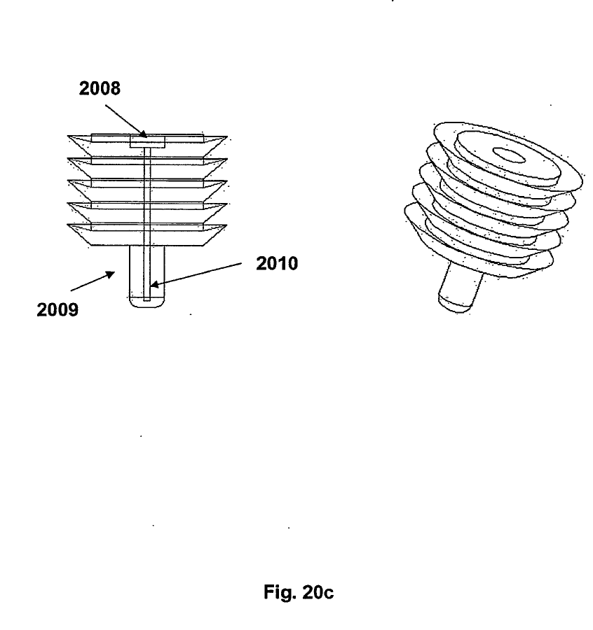

Some embodiments of the temperature sensor gasket are illustrated in FIG. 20. As shown in FIG. 20A, in one embodiment of the invention, the temperature sensor gasket is an in-stream gasket. The temperature sensor gasket features a well for sealant 2002. The fluid side of the temperature sensor gasket 2003 has an orifice 2004 at the end of a lumen that runs through the sensor through which the temperature sensor leads can be fed to contact the fluid of the system. The sensor can be mounted with sealant/adhesive for specific applications, e.g., silicone (RTV) or epoxy.

As shown in FIG. 20B, in one embodiment of the invention, the temperature sensor gasket is a mid-stream gasket. The temperature sensor gasket features a well for sealant 2005. The fluid side of the temperature sensor gasket 2006 has an orifice 2007 at the end of a lumen that runs through the sensor through which the temperature sensor leads can be fed to contact the fluid of the system for direct temperature sensing. The mid-stream gasket has an element that protrudes from the surface of the temperature sensor gasket. This allows for contact of the temperature sensor mid-stream into the fluid for direct temperature sensing. The sensor can be mounted with sealant/adhesive for specific applications, e.g., silicone (RTV) or epoxy.

As shown in FIG. 20C, in one embodiment of the invention, the temperature sensor gasket is an insulated gasket. The temperature sensor gasket features a well for sealant 2008. The fluid side of the temperature sensor gasket 2009 does not have an orifice 2010 at the end of a lumen that runs through the sensor. Rather the end of the protrusion from the gasket is sealed such that the temperature sensor leads are insulated during direct temperature sensing. The sensor can be mounted with sealant/adhesive for specific applications, e.g., silicone (RTV) or epoxy.

The temperature sensor gasket can be made of any durable material suited to its use, e.g., plastic, silicone, PVC, metal.

D. Heater-Wire Connectors of the Invention

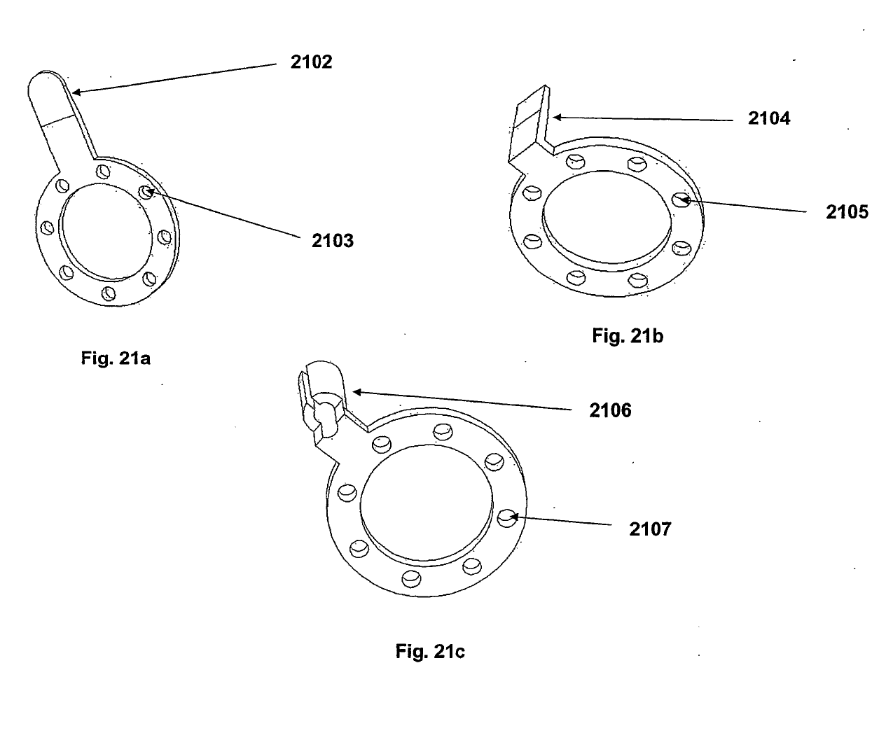

Some embodiments of the heater-wire connector are illustrated in FIG. 21. As shown in FIG. 21A, in one embodiment of the invention, the heater-wire connector has a spade terminal 2102. Leads can be attached to the spade terminal by any means of fixing a lead, e.g., a power lead, to the terminal, e.g., epoxy or solder. The heater element wire passes through the holes 2103 in the heater-wire connector. The heater-wire connector can be made of any conductive material appropriate to connect electrical elements, e.g., metal (e.g., steel, aluminum, or brass).

As shown in FIG. 21B, in one embodiment of the invention, the heater-wire connector has a spade terminal positioned at a ninety-degree angle 2104. Leads can be attached to the spade terminal positioned at a ninety-degree angle 2104 by any means of fixing a lead, e.g., a power lead, to the terminal, e.g., epoxy or solder. The heater element wire passes through the holes 2105 in the heater-wire connector. The heater-wire connector can be made of any conductive material appropriate to connect electrical elements, e.g., metal (e.g., steel, aluminum, or brass).

As shown in FIG. 21C, in one embodiment of the invention, the heater-wire connector has a crimp-style terminal 2106. Leads can be attached to the crimp-style terminal by any means of fixing a lead, e.g., a power lead, to the terminal, e.g., epoxy or solder. Alternatively, the lead can be inserted into the crimp-style terminal and crimped to secure them. The heater element wire passes through the holes 2107 in the heater-wire connector. The heater-wire connector can be made of any conductive material appropriate to connect electrical elements, e.g., metal (e.g., steel, aluminum, or brass).

In another embodiment of the invention, the heater-wire connector has a push-lock-style terminal. Leads can be attached to the push-lock-style terminal by any means of fixing a lead, e.g., a power lead, to the terminal, e.g., epoxy or solder. Alternatively, the lead can be inserted into the push-lock-style terminal and looked to secure them. The heater element wire passes through the holes in the heater-wire connector. The heater-wire connector can be made of any conductive material appropriate to connect electrical elements, e.g., metal (e.g., steel, aluminum, or brass).

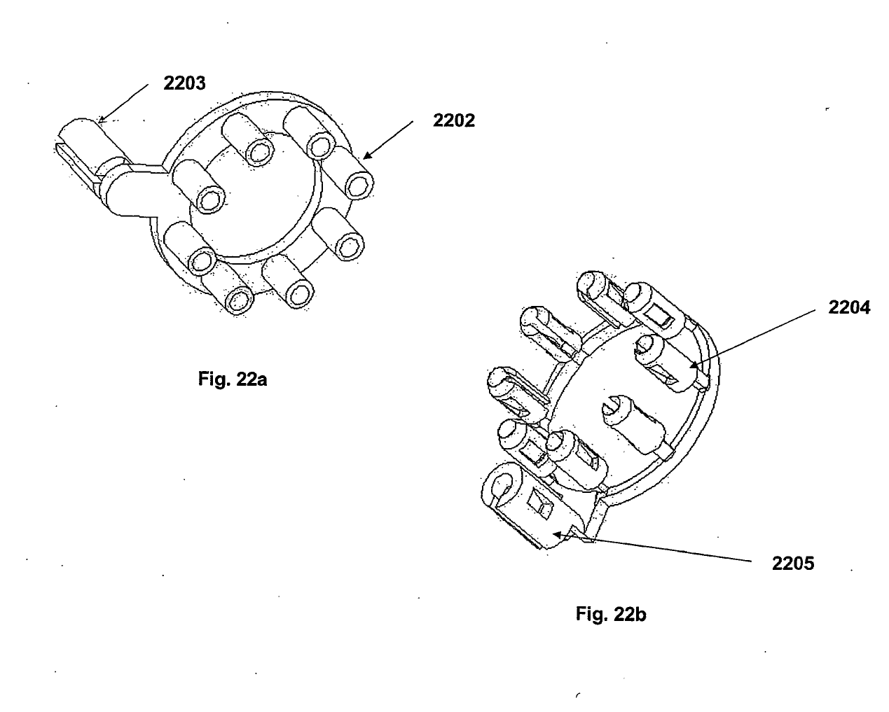

Some embodiments of the heater-wire connector are illustrated in FIG. 22. As shown in FIG. 2A, in one embodiment of the invention, the heater-wire connector has a crimp-style terminals for heater-wire elements 2202. Leads can be attached to the crimp-style terminal 2203 as described above. The heater element wire passes through the holes in the crimp-style terminals. The crimp-style terminals are contacted with the heater-wire connectors and may be crimped to secure them. The heater-wire connector can be made of any conductive material appropriate to connect electrical elements, e.g., metal (e.g., steel, aluminum, or brass).

In another embodiment of the invention, the heater-wire connector has a push-lock-style terminals for heater-wire elements 2204. Leads can be attached to the push-lock-style terminal 2205 by any means of fixing a lead, e.g., a power lead, to the terminal, e.g., epoxy or solder. Alternatively, the lead can be inserted into the push-lock-style terminal and locked to secure them. The heater element wire passes through the holes in the push-lock-style terminals. The push-lock-style terminals are contacted with the heater-wire connectors and may be push-locked to secure them. The heater-wire connector can be made of any conductive material appropriate to connect electrical elements, e.g., metal (e.g., steel, aluminum, or brass).

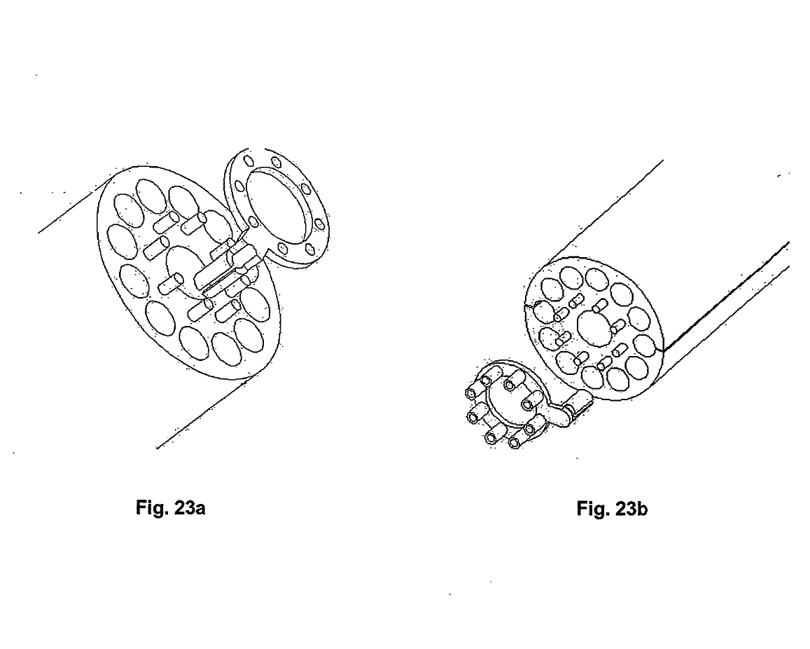

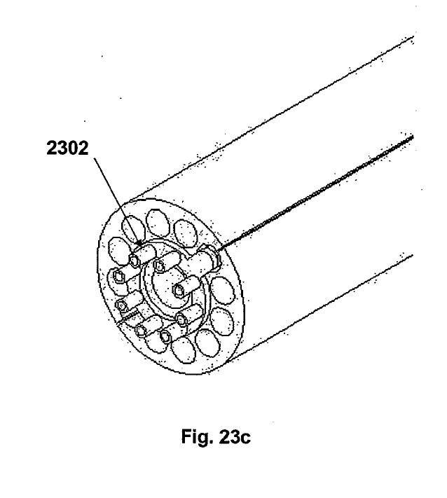

Mounting of heater-wire connectors is illustrated in FIG. 23. As shown in FIG. 23A, the solder-style connector is useful to connect exposed heater-wire elements in the fluid delivery-line component of the invention. The fluid delivery-line component may or may not have outer lumens. As shown in FIG. 23B, the heater-wire connector with a crimp-style terminals for heater-wire elements is useful to connect exposed heater-wire elements in the fluid delivery-line component of the invention. The fluid delivery-line component may or may not have outer lumens. The crimp-style terminals are contacted with the heater-wire connectors and may be crimped to secure them (FIG. 23C).

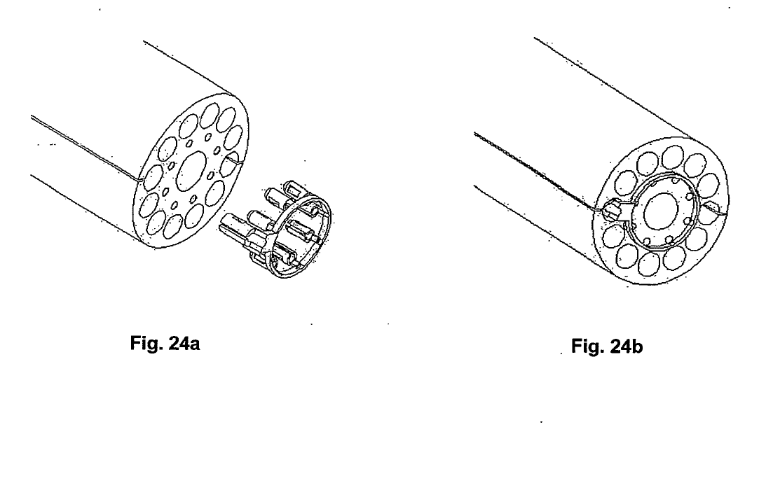

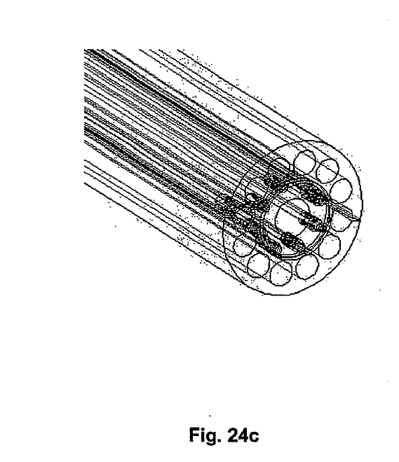

Mounting of heater-wire connectors is further illustrated in FIG. 24. As shown in FIG. 24A, the push-lock-style connector is useful to connect heater-wire elements in the fluid delivery-line component of the invention. The fluid delivery-line component may or may not have outer lumens. As shown in FIG. 24B and FIG. 24C, the heater-wire connector with a push-lock-style terminals for heater-wire elements is useful to connect heater-wire elements in the fluid delivery-line component of the invention by pressing the heater-wire connector into the fluid delivery-line component such that the push-lock-style terminals contact the heater wire elements. The fluid delivery-line component may or may not have outer lumens.

E. Temperature Sensors of the Invention

The invention provides for temperature sensing of fluid at one or more positions along the fluid delivery-line component of the system of the invention. Accordingly, designs of temperature sensors are described that can be placed in one or more positions of the fluid delivery-line component of the system of the invention for improved direct sensing of fluid temperature in the fluid delivery-line component of the system of the invention.

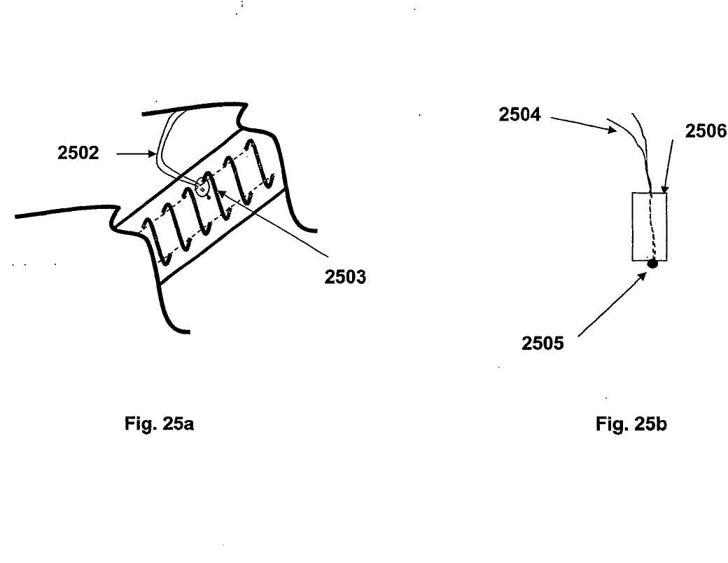

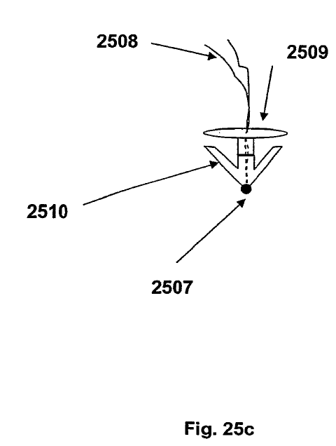

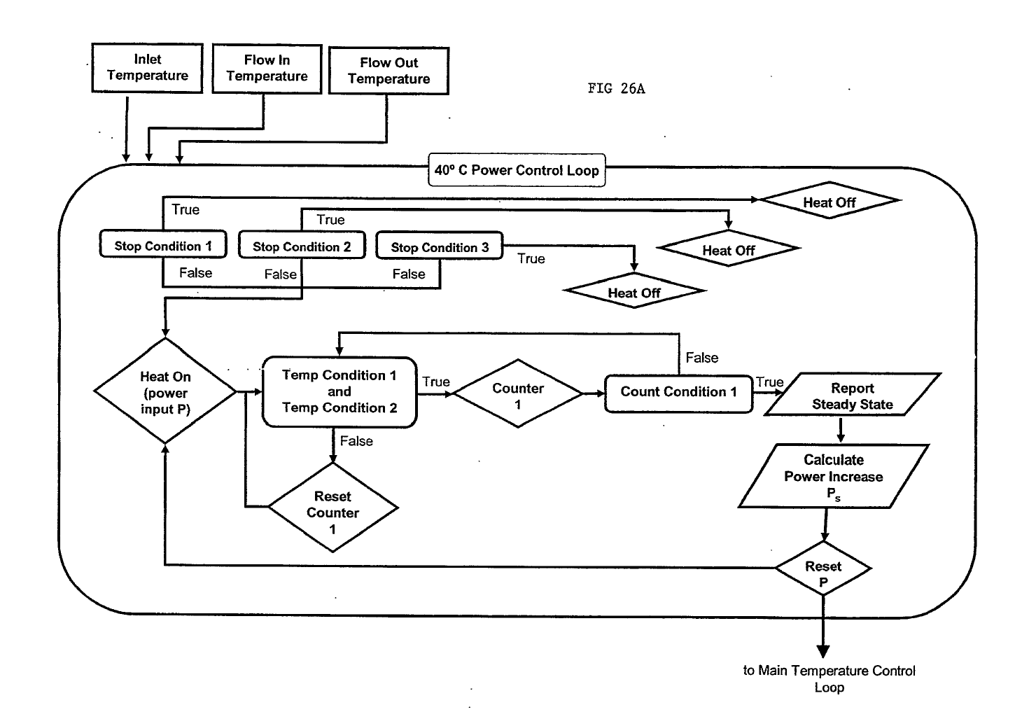

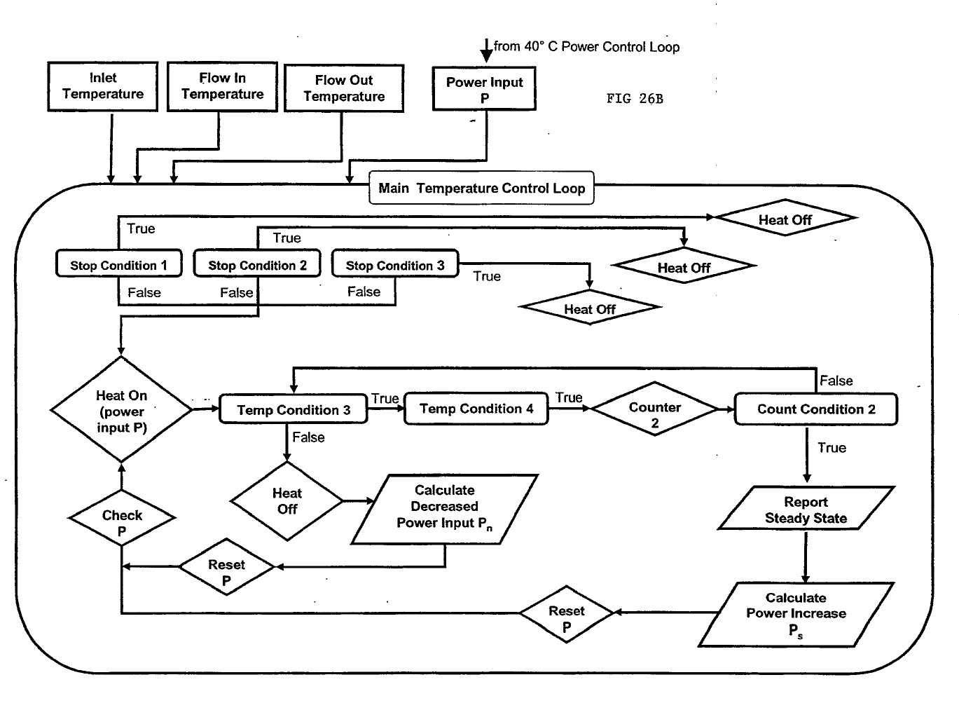

Some embodiments of the temperature sensors of the invention are illustrated in FIG. 25. As shown in FIG. 25A, in one embodiment, a temperature sensor is a center temperature sensor. A center temperature sensor is inserted through the fluid delivery-line component wall by pin piercing the wall and depositing the center temperature sensor 2502 positioned in the fluid pathway 2503 for direct temperature sensing. The leads and piercing can then be covered/secured with any appropriate sealant/adhesive, e.g., epoxy, RTV or polyolefin. In one embodiment of the invention, a pierced outer lumen in the fluid delivery-line component is used to assist in placement of the center temperature sensor.

As shown in FIG. 25B, in one embodiment of the invention, the temperature sensor is a silicone-plug-embedded-temperature sensor. As shown in FIG. 25B, silicone-plug-embedded-temperature sensor has a temperature sensor 2505 embedded in a silicone plug 2506 such that the sensor component is exposed on one of the silicone plug with the temperature sensor leads 2504 running through the silicone plug. The mid-portion of the fluid delivery-line component wall accessible via the slit can be cored for placement of the silicone-plug-embedded-temperature sensor such that the temperature sensor is contacted with the fluid stream for direct temperature sensing. The leads, plug and piercing can then be covered/secured with any appropriate sealant/adhesive, e.g., epoxy, RTV, or polyolefin. This is design is well-suited for manufacture and maintaining a low-cost disposable set.

As shown in FIG. 25C, in one embodiment of the invention, the temperature sensor is a push-pin-style temperature sensor. A push-pin-style temperature sensor is a temperature sensor that can be can be pushed through the fluid delivery-line component wall for placement of the temperature sensor in the fluid stream. As shown in FIG. 25C, a push-pin-style temperature sensor has a temperature sensor embedded in a push-pin such that the sensor component 2507 is exposed on one of the push-pin with the temperature sensor leads 2508 running through the push-pin. A push-pin-style temperature sensor has a push-in plug feature 2509 and a retaining feature 2510. In one embodiment the retaining feature of the push-pin-style temperature sensor is shaped as an arrow. The retaining feature can function to pierce the fluid delivery-line component wall and secure the push-pin-style temperature sensor. The retaining feature can be any suitable shape for piercing the fluid delivery-line component wall and securing the push-pin-style temperature sensor. The push-pin-style temperature sensor can be made of any suitable durable material, e.g., PVC or high durometer silicone. The leads, push-pin and piercing can then be covered/secured with any appropriate sealant/adhesive, e.g., epoxy, RTV, or polyolefin.

### Embodiments of the Fluid Warmer for Select Field Uses

The method and system of the present invention may be used at any suitable locations such as structured settings, emergency medical settings, and ambulatory settings, which include, but are not limited to, e.g., medical facility, emergency medical or other vehicles, or other suitable field use.

A. Use in a Structured Setting

In one embodiment useful in a structured setting such as surgical suite or patient bedside, the fluid delivery-line system is provided in a fixed axial length, for example six feet. In this embodiment, the power supply is provided by an available supply, for example an AC power outlet. The heat element configuration in this embodiment provides a maximum level of thermal control over the broadest range of fluid delivery rates. The controller in this embodiment may contain an additional input and output options, for example fluid delivery rate display or fluid type selection. The controller will also contain a memory unit for storage and recall of heating profiles and specifications.

B. Use in an Emergency Medical Setting

In another embodiment useful in a less stable environment such as a hospital trauma centers and/or emergency care facilities, the fluid delivery-line system is provided in variable axial lengths, for example three through twelve feet. In this embodiment, the power supply is variable, for example operating either AC or battery. The heating element configuration provides maximum adaptability to changing inputs and demands, such as fluid delivery-line system length and power source. The controller in this embodiment may contain additional input and output options, for example fluid rate display or fluid type selection. The controller will also contain memory unit for storage and recall of programmable information, for example audio alarm trigger values.

C. Use in an Ambulatory Setting

In another aspect of the invention, the system is useful in ambulatory applications and for use by EMT personnel in the field. In this embodiment, the fluid delivery-line system is shortened in axial length, for example thirty inches. The power supply in this embodiment is an easily portable single-use or rechargeable battery pack. The heat element configuration in this embodiment may include a heat conductive material to increase the efficiency of heating at high flow rate and short fluid delivery-line length. The controller in this embodiment may contain additional input and output options, for example fluid delivery rate display or fluid type selection. The controller in this embodiment is easily portable and conservative with power usage.

In one embodiment, the heat element is replaced with a hollow tube for circulating a coolant or a solid metallic chilling element that serves to lower the temperature of the fluid in the fluid delivery-line. This configuration may be used in the delivery of cooled fluid to a patient, for I.V. use and/or other fluid administration techniques.

In a further embodiment, one or both ends of the fluid delivery-line system terminate with bare tube in preparation for a sterile dock procedure. In another embodiment, the fluid delivery-line system is provided with one or more integral injection ports.

It should be apparent to those skilled in the art from the above descriptions of some embodiments of the present invention that the foregoing is merely illustrative and not limiting, having been presented by way of example only. Numerous modifications and other embodiments are within the scope of ordinary skill in the art and are contemplated as falling within the scope of the invention as defined by the appended claims and equivalents thereto. The contents of any references cited throughout this application are hereby incorporated by reference in their entireties. The appropriate components, processes, and methods of those documents may be selected for the invention and embodiments thereof.

### EXAMPLES

### Example 1

### Development of Algorithms Useful in the Fluid Warmer of the Present Invention

In one embodiment, the fluid warming device of the present invention uses at least two and preferably three thermocouples placed in the inner lumen at points distal, medial and proximate to the patient. The thermocouples measure the temperature of the fluid being warmed at its inlet, midpoint and outlet. The temperature is taken within the actively heated areas in all cases. An additional thermocouple measures the temperature of the heater wire. Alternatively, for a heater wire of known total resistance (determined by known wire properties and length), the heat generated can be calculated using the input current or voltage. The algorithm developed and described here is based on know parameters such as heater wire diameter, coil density per linear foot of wrap along the inner lumen, inner and outer lumen wall thickness, material and diameter. The heater wire of the invention can made of any heatable wire material, e.g., nickel-chromium or steel.

The specifications for a prototype of the fluid warmer of the present invention useful for algorithm development has the following parameters summarized below in Table 1.

TABLE 1

| Inner tube material | High Purity Silicon Rubber Tubing |
|---|---|
| Inner tube outer diameter | 0.250 inch |
| Inner tube wall thickness | 0.063 inch |
| Outer tube material | High Purity Silicon Rubber Tubing |
| Outer tube outer diameter | 0.500 inch |
| Outer tube wall thickness | 0.094 inch |
| Heater wire material | 80% Nickel 20% Chromium |
| Heater wire diameter | 0.0201 inch (24 gauge) |
| Coil density per linear foot | 72 |

The algorithm uses the data from the thermocouples, the three measuring the fluid and the optional one measuring the heater wire, to determine the heat gradient being applied to the fluid by using the fluid temperature difference from the inlet to the midpoint and the amount the heat applied by the heater wire. An analogous process is run to determine the heat gradient being applied to the fluid from the midpoint to the outlet.

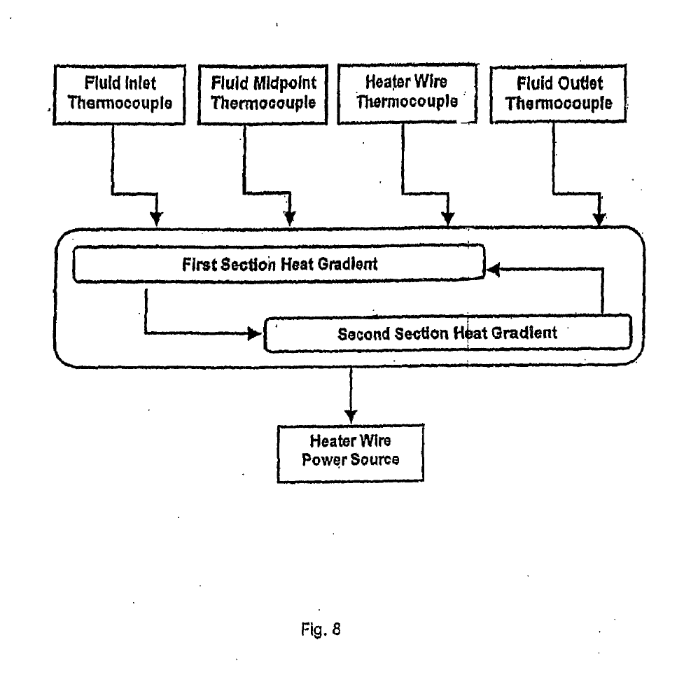

For constant flow rate the first section, defined by the inlet to midpoint, is used to modify the heat input such that the second section, defined by the midpoint to outlet, is used to generate the desired output for the entire length of the tube. In turn, the temperature at the outlet combined with the temperature at the midpoint provide actual data for comparison to the expected temperatures based on the revised heat input derived from the inlet and midpoint temperatures. Also, each thermocouple (inlet, midpoint and outlet) provides point temperature valves, which when coupled with the heater wire data, are used to determine changes in flow rate. The diagram shown in FIG. 8 illustrates the flow of data and controls for this process. The final objective of the algorithm is to iterate the heat input based on the temperature gradient from the thermocouples, such that the desired output temperature is achieved for the given fluid with minimum amount of required heat input. The reason for doing so is to maximize high flow rate capability without creating a fluid overheating condition when flow is stopped abruptly.

### Example 2

### Logic Routine and Software Development

In one embodiment, the fluid warming device of the present invention uses at least two and preferably three thermal sensors placed in the inner lumen at points distal and proximate to the patient, and on the exterior surface of the fluid supply tube. Two of the thermal sensors measure the temperature of the fluid being warmed at the heater assembly inlet and outlet. The third thermal sensor measures the exterior temperature of the fluid supply line, adjacent to the fluid supply reservoir, for extrapolation of the temperature of the fluid in the supply line at that point. The temperature is taken within the actively heated areas at two of the three sensors. The logic developed and described here is based on known parameters such as heating element resistance and initial power input.

The prototype being used for algorithm development has the following parameters:

Tube:

Custom extruded PVC with an outer diameter of 0.250 inch and a wall thickness of 0.063 inch, which features multiple, spiral run heater wires integral within the tube wall.

Software:

Custom programmed logic routines, were constructed and run on commonly available data acquisition and hardware control software for laboratory applications. Descriptions of particular set point values used during the logic and software development are listed in Table 2.

A detailed description of the logic routines including algorithms and calculations are given in FIG. 26A and FIG. 26B. The elements of FIG. 26A and FIG. 26B are defined as follows:

Inlet Temperature (T₁): From a permanent temperature sensor mounted on the control assembly, this is the continuously updated temperature value of the fluid before it enters the heating assembly.

Flow In Temperature (T₂): From a temperature sensor mounted 2″ inside the heating assembly, this is the continuously updated temperature value of the fluid before it enters the heating assembly.

Flow Out Temperature (T₃): From a temperature sensor integral to the connection fitting at the outlet end of the heating assembly, this is the continuously updated temperature value of the fluid as it exits the heating assembly.

40° C. Power Control Loop: To raise temperature of heater element to 40° C. based on the assumption that the heating assembly is empty. Known values of the heating assembly (heating element resistance) and the control assembly (initial power input), 4 ohms and 7.5 watts for example.

Stop Condition 1: Deactivates the power supply to the heating element in the case when the Flow In Temperature (T₂) exceeds the set point value. Stop Condition 1 prevents the active heating of a fluid that is at a suitable temperature, 30° C. for example, when it enters the heating assembly. Stop Condition 1 also improves power management in low flow rate heating applications.

Stop Condition 2: Deactivates the power supply to the heating element in the case when the Inlet Temperature (T₁) is subtracted from the Flow In Temperature (T₂) and the remainder exceeds a set point value, 1.0 for example. Stop Condition 2 detects a stop flow or extreme flow decrease event and prevents active heating during the event. Stop Condition 2 also detects the presence of air bubbles in the fluid flow and deactivates power to the heating element.

Stop Condition 3: Deactivates the power supply to the heating element in the case when the Flow Out Temperature (T₃) exceeds a set point value. Stop Condition 3 provides top level protection from overheating, by preventing the active heating of fluid in the heating assembly when the fluid at the outlet is at a suitable temperature, 40° C. for example.

Temp Condition 1: T₃>(T₂+C_{fa}), where C_{fa} is the constant, known as the “flow-in adder”. All values are integers.

Temp Condition 2: T₃ (time=t)=T₃ (time=t−1).

Counter 1: Function that holds and augments a count variable (n) by a set value on each operation.

Count Condition 1: When the count variable (n) equals a set point value, 10 for example, report that the value of T₃ has reached a steady state.

Count Condition 2: Yields optimal power increase value (Pₛ), in watts, for existing fluid temperatures and target fluid output temperature (T_{target}).

$${P}_{s} = [ \frac{( {T}_{target} - {T}_{3} )}{{T}_{divisor}} ] [ \frac{{P}_{initial}}{{T}_{3} - {T}_{2}} ] [ {T}_{target} - {T}_{3} ]$$

where T_{divisor} is target differential divisor and Pᵢₙᵢₜᵢₐₗ is the initial power input.

Reset P: Sets the value of the power input variable (P) equal to the newly calculated value. Also sends the newly calculated power input value to the Main Temperature Control Loop, which initiates its function and ceases the operation of the 40° C. Power Control Loop.

Main Temperature Control Loop: To monitor and maintain temperature of fluid in heater assembly.

Stop Condition 4: Same as 1. To deactivate power to the heating element in the case where the Flow In Temperature (T₂) exceeds a set point value. Stop Condition 4 prevents the active heating of a fluid that is at a suitable temperature, 30° C. for example, when it enters the heating assembly. Stop Condition 4 also improves power management in low flow rate heating applications.

Stop Condition 5: Same as 2. To deactivate power to the heating element in the case where the Inlet Temperature (T₁) subtracted from the Flow In Temperature (T₂) exceeds a set point value, 1.0 for example. Stop Condition 5 detects a stop flow or extreme flow decrease event and prevents active heating during the event.

Stop Condition 6: Same as 3. To deactivate power to the heating element in the case where the Flow Out Temperature (T₃) exceeds a set point value. Stop Condition 6 provides top level protection from overheating, by preventing the active heating of fluid in the heating assembly when the fluid at the outlet is at a suitable temperature, 40° C. for example.

Temp Condition 3: T₃≦T_{target}

Temp Condition 4: T₃ (time=t)=T₃ (time=t−1)

Counter 2: Function that holds and augments a count variable (n) by a set value on each operation.

Count Condition 2: When the count variable (n) equals a set point value (preset stop value), 10 for example, report that the value of T₃ has reached a steady state.

Calculate Power Increase Pₛ: Yields optimal power increase value (Pₛ), in watts, for existing fluid temperatures and target fluid output temperature (T_{target}).

$${P}_{s} = [ \frac{( {T}_{target} - {T}_{3} )}{{T}_{divisor}} ] [ \frac{{P}_{initial}}{{T}_{3} - {T}_{2}} ] [ {T}_{target} - {T}_{3} ]$$

Where T_{divisor} is target differential divisor and Pᵢₙᵢₜᵢₐₗ is the initial power input.

Calculate Power Decrease Pₙ: Yields a new (decreased) power input value (Pₙ), in watts, based on current power input (P).

Pₙ=(P)−(P/Cₜᵣ), where Cₜᵣ is the constant, known as the “temperature reduction divisor”.

Reset P: Sets the value of the power input variable (P) equal to the newly calculated power input value.

Check P: In the case where the newly reset power input (P) is less than initial system power (Pᵢ), the value of the power input (P) is set equal to the initial power (Pᵢ). During a power decrease event, a sudden decrease in flow rate for example, Check P prevents the power input from being reduced to less than the known minimum power input, improving power management.

The first routine uses the data from the two thermal sensors at the inlet and outlet of the heating assembly and the one thermal sensor on the exterior of the fluid supply line to determine the amount of heating for a known power input. This routine utilizes an algorithm developed specifically for this application, that generates a required power input based upon the individual flow rate and temperature values for each fluid transfused. (See FIG. 26A) Once the fluid temperature reaches a steady state and the power input increase is calculated, the first routine initiates the second routine. (See FIG. 26B)

The second routine uses data from the same three thermal sensors and from the first routine to achieve the desired fluid temperature and maintain said fluid temperature, despite changes in flow rate. (FIG. 26B) This routine utilizes the specifically developed algorithm that generates a required power input based upon the individual flow rate and temperature values for each fluid transfused. In addition the second routine performs checks and calculations for power decrease event, stop flow or drastic flow reduction for example. The second loop is optimized for extended fluid temperature management using variable power input.

TABLE 2
User Defined Software Set Points

| Descriptor | Name | Value (range) | |
|---|---|---|---|
| Stop Condition 1: Flow In Temperature (T2) set | flowin max | 30° C. | (30-35) |
| point value | | | |
| Stop Condition 2: Inlet Temperature (T1) - Flow In | flowin-inlet | 1° | (0.1-2.0) |
| Temperature (T2) set point value | | | |
| Stop Condition 3: Flow Out Temperature (T3) set | flowout max | 40° C. | (40-42) |
| point value | | | |
| Counter 1: count initial value | preset start | 1 | |
| | value | | |
| Count Condition 1: count variable (n) set point | preset stop | 10 | |
| value | value | | |
| Stop Condition 3: Flow In Temperature (T2) set | flowin max | 30° C. | (30-35) |
| point value | | | |
| Stop Condition 4: Inlet Temperature (T1) - Flow In | flowin-inlet | 1° | (0.1-2.0) |
| Temperature (T2) set point value | | | |
| Stop Condition 5: Flow Out Temperature (T3) set | flowout max | 40° C. | (40-42) |
| point value | | | |
| Counter 2: count initial value | preset start | 1 | |
| | value | | |
| Count Condition 2: count variable (n) set point | preset stop | 10 | |
| value | value | | |
| flowin adder | C_{fa} | 3° C. | (1-10) |

| | | |
|---|---|---|
| temperature reduction divisor | Cₜᵣ | 10 |

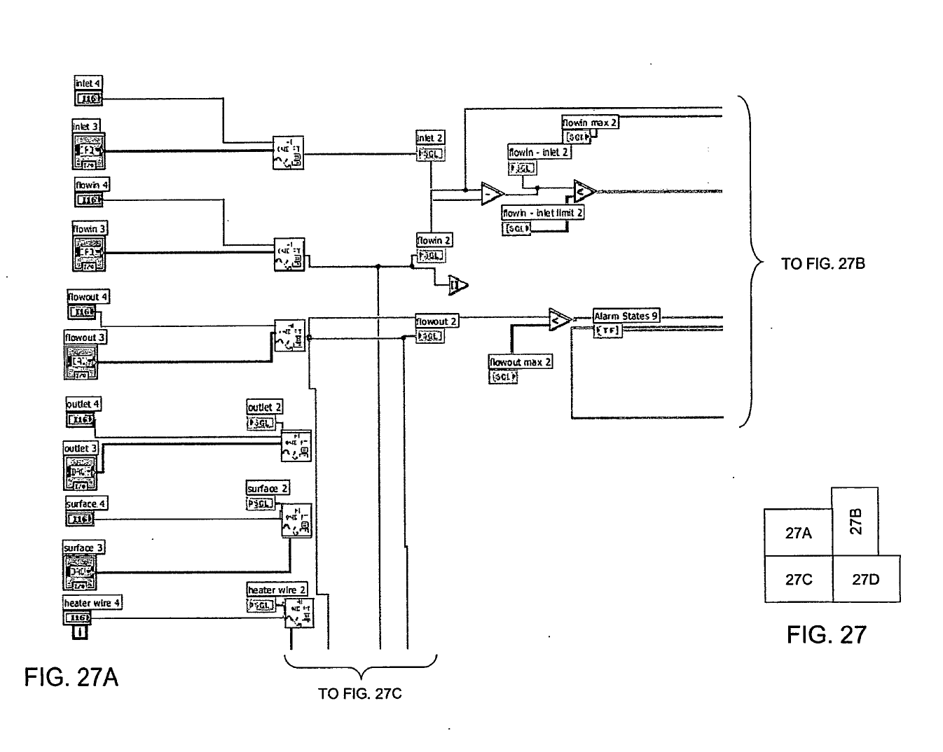

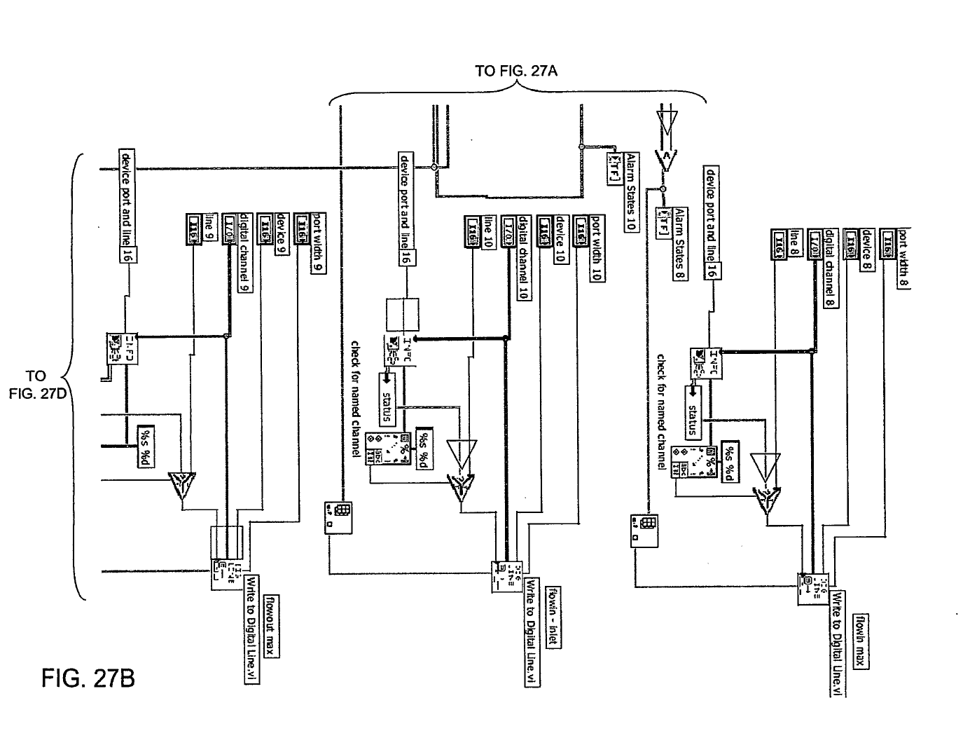

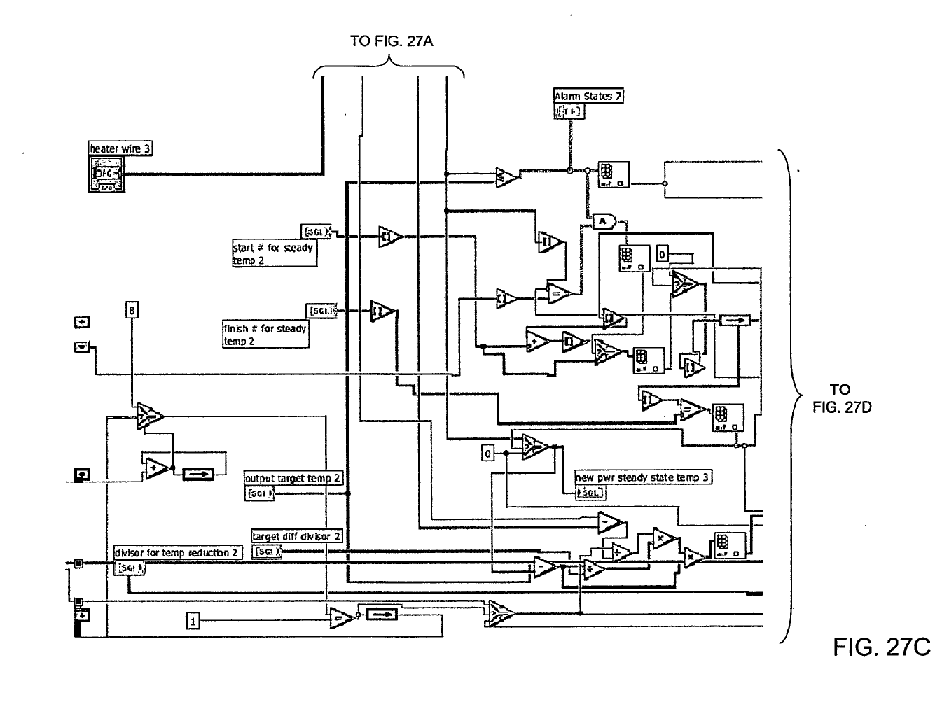

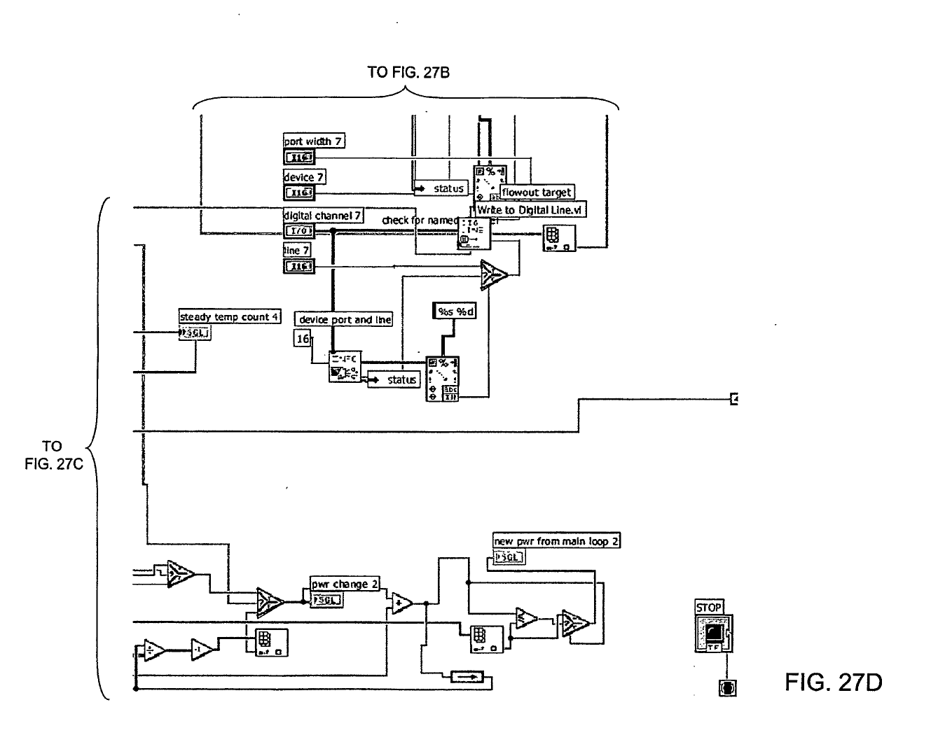

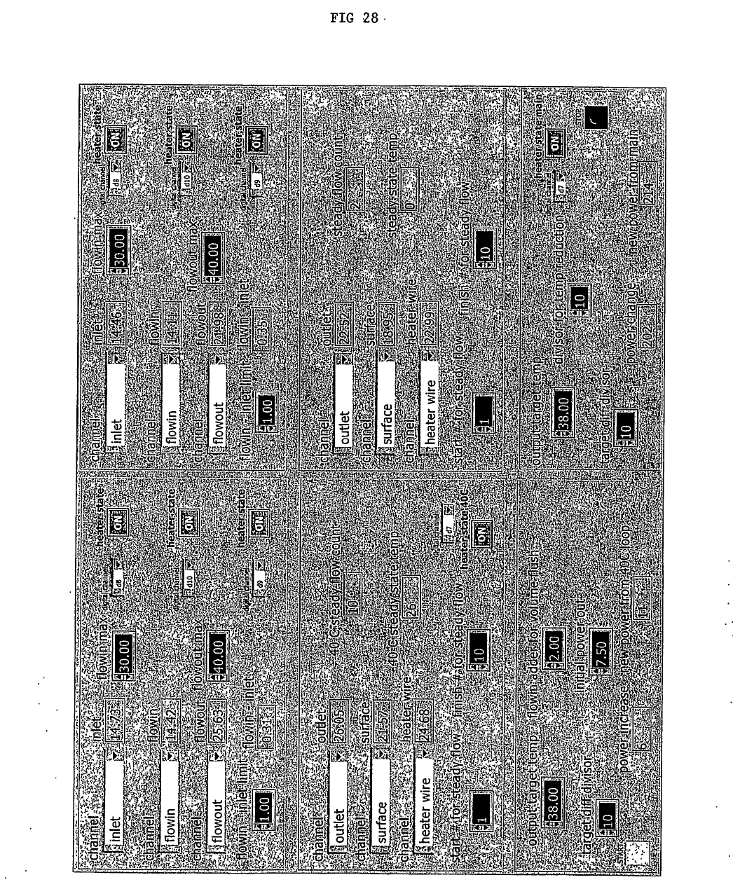

Detailed description of the block and the panel are given in FIG. 27 and FIG. 28, respectively.

### EQUIVALENTS

From the foregoing detailed description of the invention, it should be apparent that a unique method and system for warming a fluid have been described resulting in improved fluid warming suitable for administration to a patient. Although particular embodiments have been disclosed herein in detail, this has been done by way of example for purposes of illustration only, and is not intended to be limiting with respect to the scope of the appended claims, which follow. In particular, it is contemplated by the inventor that substitutions, alterations, and modifications may be made to the invention without departing from the spirit and scope of the invention as defined by the claims. For instance, the choice of fluid delivery-line component length, fluid delivery-line component style, fluid flow rate, fluid temperature, as well as the number and positioning of the temperature sensors is believed to be matter of routine for a person of ordinary skill in the art with knowledge of the embodiments described herein.

What is claimed is:

1. A system for heating a fluid for delivery into a body of a patient comprising:
a flexible fluid supply reservoir;
a controller; and a fluid delivery-line comprising:
a fluid delivery tube for communicating a fluid;
a heating element positioned proximate a surface of the fluid delivery-line to heat fluid within the tube;
a first thermal sensor positioned on the exterior of the fluid delivery tube and adjacent to the fluid supply reservoir; and
a second thermal sensor positioned at a heater assemble outlet and in communication with the lumen of the fluid delivery tube.

2. The system of claim 1, wherein the fluid delivery-line further comprises a third thermal sensor positioned at a heater assembly inlet and in communication with a lumen of the fluid delivery tube.

3. The system according to claim 1, wherein the heating element is spaced apart from an outer surface of the second tube.

4. The system according to claim 1, wherein a wall of the tube comprises a thermal medium for distributing heat received by the outer surface of the tube from the heating element.

5. The system according to claim 1, wherein the heating element surrounds the tube.

6. The system according to claim 1, wherein the heating element spirally surrounds the tube.

7. The system according to claim 1, wherein the heating element comprises a plurality of heating elements surrounding the tube and having a length positioned substantially parallel to a length of the tube.

8. The system according to claim 1, wherein the heating element comprises a plurality of heating elements, each circumferentially surrounding the tube and spaced apart from one another along a length of the tube.

9. The system according to claim 1, wherein the heating element is surrounded by a thermal medium.

10. The system according to claim 1, wherein the thermal medium comprises a fluid.

11. The system according to claim 1, wherein the fluid delivery tube includes a bag spike positioned at one end.

12. The system according to claim 1, wherein the fluid delivery tube includes a transfusion needle and/or a leur-lock at one end.

13. The system according to claim 1, wherein the heating element and/or thermal sensor are in electrical contact with the controller.

14. The system according to claim 1, wherein the controller is connected to a power source.

15. The system according to claim 1, wherein the power source is selected from the group consisting of: a one-time use battery pack, a rechargeable battery pack, AC power, and DC power.

16. The system according to claim 1, wherein the tube is sterile prior to use.

17. The system according to claim 1, wherein the controller provides an electrical current to the heating element.

18. The system according to claim 17, wherein the controller controls the temperature of the fluid delivery-line tube by sensing a temperature corresponding to a temperature of fluid within the fluid delivery-line and adjusting the amount of current supplied to the heating element to maintain the temperature of the fluid independent of the flow rate of the fluid.

19. The system according to claim 1, further comprising a heat element connector and/or a thermal sensor connector for connecting the heat element and thermal sensor, respectively, to corresponding connectors on the controller.

20. The system according to claim 1, further comprising a valve.

21. The system according to claim 20, wherein the valve comprises a temperature actuated valve that opens upon the temperature of the fluid within the second tube reaching a predetermined value.

22. The system according to claim 1, further comprising a metering means for determining a flow rate of fluid traversing through the fluid delivery tube.

23. The system according to claim 1, further comprising a heat-conductive member having a first portion placed adjacent an interior portion of the fluid delivery tube and a second portion placed proximate the heating element, wherein the heat-conductive material transfers heat from the heating element to the interior portion of the fluid delivery tube.

24. The system according to claim 1, further comprising an insulative tube, wherein the fluid delivery tube is positioned within the insulative tube.

25. The system according to claim 24, further comprising a thermal medium positioned between the fluid delivery tube and the insulative tube.

26. The system according to claim 25, wherein the thermal medium envelops the heating element.

27. A method of heating a fluid for delivery into the body of a patient comprising:
providing a fluid delivery tube having a first end for connection to a flexible fluid supply reservoir; a controller; and a fluid delivery-line comprising a fluid source and a second end for delivering the fluid from the fluid source to a destination;
applying an electrical current to a heating element proximate to and/or within the fluid delivery tube to heat fluid therein to a predetermined temperature;
sensing, via a first thermal sensor positioned on the exterior of the fluid delivery tube and adjacent to a flexible fluid supply reservoir and a second thermal sensor positioned at a heater assemble outlet and in communication with the lumen of the fluid delivery tube, a temperature corresponding to the temperature of the fluid within the tube; and
adjusting the current applied to heating element via the controller based upon the sensed temperature.

28. The method of claim 27, wherein the fluid delivery-line further comprises a third thermal sensor positioned at a heater assembly inlet and in communication with a lumen of the fluid delivery tube.

29. The method according to claim 28, wherein the current is decreased or stopped upon the temperature of the fluid delivery tube reaching the predetermined temperature.

30. The method according to claim 29, further comprising opening a valve which controls the movement of fluid from the fluid delivery-line to the patient upon the temperature of the fluid for delivery reaching the predetermined temperature.

31. The method according to claim 29, further comprising sensing a flow-rate of the fluid being delivered to the patient.

31. A system for heating a fluid for delivery into the body of a patient comprising:
a flexible fluid supply reservoir;
a controller; and
a fluid delivery-line comprising:
an insulative tube;
a fluid delivery tube positioned within the first tube, the fluid delivery tube for communicating a fluid;
a fluid delivery-line having a first end for receiving fluid from a fluid source and delivering the fluid to a destination, the fluid delivery-line comprising:
a heating element positioned proximate a surface of the fluid delivery-line to heat fluid within the tube;
a first thermal sensor positioned on the exterior of the fluid delivery tube and adjacent to the fluid supply reservoir; and
a second thermal sensor positioned at a heater assemble outlet and in communication with the lumen of the fluid delivery tube; and
a thermal medium positioned between the first tube and the second tube.

32. The system of claim 31, wherein the fluid delivery-line further comprises a third thermal sensor positioned at a heater assembly inlet and in communication with a lumen of the fluid delivery tube.
